(19) United States
(12) Patent Application Publication
(10) Pub. No.: US 2010/0190832 A1
(43) Pub. Date: Jul. 29, 2010

(54) Mechanism-based inhibitors of transthyretin amyloidosis: studies with biphenyl ethers and structural templates

(75) Inventor: Avadhesha Surolia, New Delhi (IN)

Correspondence Address: LADAS & PARRY LLP, 26 WEST 61ST STREET, NEW YORK, NY, 10023, US

(73) Assignee: NATIONAL INSTITUTE OF IMMUNOLOGY.

(21) Appl. No.: 12/587,990

(22) Filed: Oct. 15, 2009

Related U.S. Application Data
(60) Provisional application No. 61/196,453, filed on Oct. 17, 2008.

(51) Int. Cl. A61K 31/09; A61K 31/192; A61K 31/11; A61K 31/4245; A61K 31/4196; A61K 31/198; A61P 25/28; A61P 3/00; A61P 25/00

(52) U.S. Cl. 514/364; 514/717; 514/721; 514/568; 514/699; 514/383; 514/567

(57) ABSTRACT

Transthyretin (TTR), a tetrameric thyroxine (T4) carrier protein, is associated with a variety of amyloid diseases. Derivative of biphenyl ethers (BPE), which are shown to interact with a high affinity to its T4 binding site thereby preventing its aggregation and fibrillogenesis. They prevent fibrillogenesis by stabilizing the tetrameric ground state of transthyretin. Two compounds (2-(5-mercapto-[1,3,4]oxadiazol-2-yl)-phenol and 2,3,6-trichloro-N-(4H-[1,2,4]triazol-3-yl) exhibit the ability to arrest TTR amyloidosis. The dissociation constants for the binding of BPEs and compound 11 and 12 to TTR correlate with their efficacies of inhibiting amyloidosis. They also have the ability to inhibit the elongation of intermediate fibrils as well as show nearly complete (>90%) disruption of the preformed fibrils. Biphenyl ethers and compounds 11 and 12 as very potent inhibitors of TTR fibrillization and inducible cytotoxicity.

### BACKGROUND OF THE INVENTION

Protein misfolding, misassembly, and extracellular deposition are related to a class of diseases collectively known as “conformational diseases”, which include Alzheimer's disease (Kisilevsky, R. Amyloid beta threads in the fabric of Alzheimer's disease. Nat. Med. 1999, 4, 772-773), prion disease (Harrison, P. M.; Bamborough, P.; Daggett, V.; Prusiner, S. B.; Cohen, F. E. The prion folding problem. Curr. Opin. Struct. Biol. 1997, 7, 53-59), dialysis-related amyloidosis, (Reese, W.; Hopkovitz, A.; Lifschitz, M. D. B2-microglobulin and associated amyloidosis presenting as bilateral popliteal tumors. Am. J. Kidney Dis. 1988, 12 (4), 323-325), familial amyloid polyneuropathy (Kelly, J. W., et al. Structure 1997, 5, 595-600) and type II diabetes Westermark, P.; Wernstedt, C.; (Wilander, E.; Hayden, D. W.; O'Brien, T. D.; Johnson, K. H. Amyloid fibrils in human insulinoma and islets of Langerhans of the diabetic cat are derived from a neuropeptide-like protein also present in normal islet cells. Proc. Natl. Acad. Sci. U.S.A. 1987, 84, 3881-3885). Most of these diseases are incurable and fatal. Proteins and peptides related to these diseases can self-assemble into supramolecular assemblies with a common cross-β structure. Despite a large variation in their sequences and native structures, they adopt a similar morphology upon fibril formation, which suggests that there is a common mechanism underlying amyloid fibril formation. (Dabson, C. M. Protein misfolding, evolution and disease. Trends Biochem. Sci. 1999, 9, 329-332.)

Transthyretin (TTR^{a}), a tetrameric protein, transports thyroxine and holo retinol binding protein in plasma and cerebrospinal fluid (Hamilton, J. A.; Benson, M. D. Protein misfolding, evolution and disease. Cell Mol. Life. Sci. 2000, 58, 1491-1521). Further, it also scavenges Aβ peptide, preventing its aggregation and thereby regulates the pathogenesis of Alzheimer's disease (Lin Liu Murphy, R. M. Kinetics of Inhibition of β-Amyloid Aggregation by Transthyretin. Biochemistry 2006, 45, 15702-15709). TTR tetramer has a tendency to dissociate to unfolded monomers, which aggregate together resulting in the formation of amyloid fibers, which constitute the hallmark of familial amyloid cardiomyopathy (FAC), senile systemic amyloidosis (SSA, late onset), and familial amyloid polyneuropathy (FAP, early onset). In SSA and FAC, wild type TTR forms amyloid deposits on the cardiac and other tissues (Gustavsson, A.; Jahr, H.; Tobiassen, R.; Jacobson, D. R.; Sletten, K.; Westermark, P. Amyloid fibril composition and transthyretin gene structure in senile systemic amyloidosis. Lab. Invest. 1995 73 (5), 703-708 and Saraiva, M. J. Transthyretin mutations in health and disease. Hum. Mutat. 1995, 5 (3), 191-196). Approximately 100 mutants have been identified in TTR to be involved in FAP, which affect the peripheral and autonomic nervous system, heart, and the CNS. (Vidal, R.; Garzuly, F.; Budka, H.; Lalowski, M.; Linke, R. P.; Brittig, F.; Frangione, B.; Wisniewski, T. Meningocerebrovascular amyloidosis associated with a novel transthyretin mis-sense mutation at codon 18 (TTRD 18G). Am. J. Pathol. 1996, 148, 361-366 and Hammarström, P.; Sekijima, Y.; White, J. P.; Wiseman, R. L.; Lim, A.; Costello, C. E.; Altland, K.; Garzuly, F.; Budka, H.; Kelly, J. W. D18G transthyretin is monomeric, aggregation prone, and not detectable in plasma and cerebrospinal fluid: a prescription for central nervous system amyloidosis? Biochemistry 2003, 42, 6656-6663).

Irrespective of the types of TTR (viz. wild type TTR or its mutants) involved in amyloid formation, the overall mechanism of fibril formation is similar. The common mechanism involves the dissociation of the tetramer into non-native monomers, which eventually agglomerate into fibers. TTR deposits are predominantly extracellular in nature, while some of its variants also exhibit tissue specificity (Jacobson, D. R.; Pastore, R. D.; Yaghoubian, R.; Kane, I.; Gallo, G.; Buck, F. S.; Buxbaum, J. N. Variant-sequence transthyretin (isoleucine 122) in late-onset cardiac amyloidosis in black Americans. N. Engl. J. Med. 1997, 336, 466-473). The mechanism for their tissue selectivity and the pathway of their deposition in vivo are as yet poorly understood.

The quaternary structure of TTR contains two funnel shaped thyroxine (T4) binding sites. Blake, C. C.; Geisow, M. J.; Oatley, S. J.; Rerat, B.; Rerat, C. F. Structure of prealbumin: secondary, tertiary and quaternary interactions determined by Fourier refinement at 1.8 A. J. Mol. Biol. 1978, 121, 339-356). Under physiological conditions, only 10-25% of T4 in the plasma is bound to TTR. (Bartalena, L.; Robbins, J. Thyroid hormone transport proteins. Clin. Lab. Med. 1993, 13, 583-598).

The stabilization of TTR tetramer by small molecules, which bind to the T4 pocket, is an emerging theme in a number of studies aiming to stall amyloidogenic potential of TTR (Hammarström, P.; Schneider, F.; Kelly, J. W. Trans-suppression of misfolding in an amyloid disease. Science 2001, 293, 2459 and Hammarstrom, P.; Wiseman, R. L.; Powers, E. T.; Kelly, J. W. Prevention of transthyretin amyloid disease by changing protein misfolding energetics. Science 2003, 299, 713-716). So far, a number of TTR amyloidosis inhibitors including both natural and synthetic molecules that span a variety of structural classes have met with limited success. (Morais-de-Sa, E. et al.; Acta Crystallogr. D Biol. Crystallogr. 2006, 62 (5), 512-519; Morais-de-Sa, E.; Pereira et al., J. Biol. Chem., 2004, 279, 53483-53490; Gales, L. et al. Biochem. J. 2005, 388, 615-621; Green, N. S. et al.; Proc. Natl. Acad. Sci. U.S.A. 2005, 102, 14545-14550; Green, N. S. et al.; J. Am. Chem. Soc. 2003, 125, 13404-13414. Johnson, S. M. et al.; J. Med. Chem. 2005, 48, 1576-1587; Petrassi, H. M. et al.; J. Am. Chem. Soc. 2005, 127, 6662-6671; Petrassi, H. M. et al.; J. Am. Chem. Soc. 2000, 122, 2178-2192; Adamski-Werner, S. L. et al.; J. Med. Chem. 2004, 47, 355-374; Almeida, M. R et al.;. Biochem. J. 2004, 381, 351-356; Miller, S. R. et al.; Lab. Invest. 2004, 84, 545-552; Razavi, H. et al.; Angew. Chem. 2003, 42, 2758-2761; Cardoso, I. et al.; FASEB J. 2003, 17, 803-809; Sebastiao, M. P. et al.; Biochem. J. 2000, 351, 273-279; Oza, V. B. et al.; J. Med. Chem. 2002, 45, 321-332; Raghu, P. et al.; Arch. Biochem. Biophys. 2002, 400 (1), 43-47; Klabunde, T. et al.; Nat. Struct. Biol. 2000, 7, 312-321. Peterson, S. A. et al.; Proc Natl Acad Sci U.S.A. 1998, 95, 12956-12960; Baures, P. W. et al.; Bioorg. Med. Chem. 1998, 6, 1389-1401 and Miroy, G. J.; et al.; Proc. Natl. Acad. Sci. U.S.A. 1996, 93, 15051-15056.)

Several reports indicate that inhibition of fibril formation can lead to accumulation of soluble prefibrillar oligomeric intermediates, the most cytotoxic species. Reixach, N. et al.; Proc. Natl. Acad. Sci. U.S.A. 2004, 101, 2817-2822 and Sousa, M. M.; Cardoso, I. et al; J. Am. J. Pathol. 2001, 159, 1993-2000.)

Chemical modification and covalent linking of molecules to TTR have also been suggested as alternatives to these approaches. (Erlanson, D. A. et al.; Curr. Opin. Chem. Biol. 2004, 8, 399-406; Altland, K.; et al., M. J.; Suhr, O, Sulfite and base for the treatment of familial amyloidotic. Neurogenetics 2004, 5, 61-67 and Altland, K et al.; Neurogenetics 1999, 2, 183-188).

The most promising approach includes stabilization of the native state of these proteins, which is best exemplified by TTR amyloidosis. Earlier reports have demonstrated that native state kinetic stabilization is a viable therapeutic approach to prevent TTR amyloidosis. Weisman, L. R.; Johnson, S. M.; Kelker, M. S.; Foss, T.; Wilson, I. A.; Kelly, J. W. Kinetic stabilization of an oligomeric protein by a single ligand binding event. J. Am. Chem. Soc., 2005, 127, 5540-5551).

Biphenyl ether (BPE) as a template to design potential inhibitors of TTR amyloidosis has not yet been explored systematically. The structure-activity relationship (SAR) studies have shown that small molecule such as triclosan, could inhibit TTR aggregation. (Dolado, I.; Nieto, J.; Saraiva, M. J.; Arsequell, G.; Valencia, G.; Planas, A. Kinetic assay for high-throughput screening of in vitro transthyretin amyloid fibrillogenesis inhibitors. J. Comb. Chem. 2005, 7, 246-252).

Triclosan, which has a biphenyl ether skeleton, resembles T4 in its gross structure.

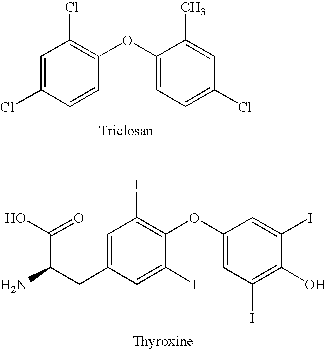

Triclosan exhibits a wide range of pharmacological properties including antibacterial and antimalarial activities. (Surolia, N.; Surolia, A. Triclosan offers protection against blood stages of malaria by inhibiting enoyl-ACP reductase of Plasmodium falciparum. Nat. Med. 2001, 7, 167-173).

### SUMMARY OF THE INVENTION

The present invention relates to a method of treating, modulating or preventing an amyloid-related disease (amyloidosis).

The present invention relates to the use of derivatives of biphenylether (BPE) in the treatment of amyloid related diseases (amyloidosis).

Another aspect of the invention relates to a method of treating, modulating or preventing an amyloid-related disease in a subject comprising administering to the subject a therapeutic amount of a compound that is derivative of a BPE.

In still another aspect of the invention, the derivatives of BPE compounds disclosed herein prevent or inhibit amyloid protein assembly into insoluble fibrils which, in vivo, are deposited in various organs.

The invention also pertains to pharmaceutical compositions for the treatment, modulation or prevention of amyloid-related diseases.

### BRIEF DESCRIPTION OF THE FIGURES

FIG. 1 shows the semilog of TTR (7.2 μM) stagnant acid-mediated fibrillazation assay at pH 4.4 in the presence of compounds 1-12 and thyroxine at various concentrations over 72 h.

FIG. 2 (a) is a comparative bar diagram of turbidity (shaded bars) and quantitative Congo red binding (black bars) assay to quantitate TTR (7.2 μM) fibrillogenesis in the absence and presence of 3 equiv (21.6 μM) of compounds 1-12 and T4.

FIG. 2(b) shows the time course of TTR (7.2 μM) fibril formation in the presence and absence of compounds 1-12 (shown in Table 2) and T4 (21.6 μM) at 37° C., pH 4.4.

FIG. 2(c) shows Thioflavin-T fluorescence after binding to TTR (7.2 μM) in the presence and absence of compounds 1-12 (shown in Table 2) and T4 (21.6 μM) under fibrillization condition.

FIG. 3(a) and FIG. 3(b) show the conformational change in TTR induced by binding of compounds 1-12 (shown in Table 2) and T4. FIG. 3(a) shows the changes at pH 7.2, and FIG. 3(b) at pH 4.4.

FIG. 3(c) Time dependence of tetramer dissociation (fraction unfolded) of TTR in the presence and absence of inhibitors in 6 M urea at 25° C.

FIG. 4(a) shows oligomeric status of TTR in the presence and absence of compounds 1-12 (shown in Table 2) at pH 4.4 after 15 days of incubation under fibrillization conditions.

FIG. 4(b) shows stability of TTR treated with compounds 1-12 (shown in Table 2).

FIG. 4(c) shows efficacies of compounds 2-12 (shown in Table 2) in protecting Neuro2a cells against cytotoxicity of TTR.

FIG. 5(a), (b) and (c) show the results of the docking of compounds 2-12 (shown in Table 2) on the TTR tetramer.

FIG. 6(a) shows Th-T fluorescence of TTR fibers after disruption by compounds 2-12 (shown in Table 2).

FIG. 6(b) shows Native-PAGE analysis of samples of TTR fibers disruption after 1 month of incubation with compounds 2-12 at 37° C.

FIG. 6(c) shows transmission electron micrograph of control sample showing fibrillar aggregates (1:2 diluted, 4.2 K).

FIG. 6(d) shows control sample showing full length fibers (1:100 diluted, 8.2 K).

FIG. 6(e) shows fibers incubated with compounds 2-12 (shown in Table 2) for 2 days were clearly disrupted (16.5 K).

FIG. 6(f) shows magnified view of disrupted fibers (87 K).

FIG. 7(a)-(j) are transmission electron micrographs of TTR fibers.

FIG. 8 is a plot that shows the rate of fibril formation in the presence and absence of compounds 1-12 (shown in Table 2) and T4 is preventing TTR fibril formation.

FIG. 9(a)-(c) shows the mass spectra of TTR complexed with inhibitors 1-12 (shown in Table 2).

FIG. 10(a)-(g) show inhibition of fiber elongation and disruption of preformed TTR fibers by compounds 2-12 (shown in Table 2).

### DETAILED DESCRIPTION OF THE INVENTION

The present invention relates to the use of derivatives of BPE compounds in the treatment, modulation or prevention of amyloid-related diseases. The invention relates to a method of treating or preventing an amyloid-related disease in a subject (for example, a human) comprising administering to the subject a therapeutic amount of a compound as described herein, such that amyloid fibril formation or deposition, or cellular toxicity is reduced or inhibited.

The compounds of the invention may be administered therapeutically or prophylactically to treat diseases associated with amyloid β fibril formation, aggregation or deposition. The compounds of the invention may act to ameliorate the course of an amyloid β related disease using any of the following mechanisms (this list is meant to be illustrative and not limiting): slowing the rate of amyloid β fibril formation or deposition; lessening the degree of amyloid β deposition; inhibiting, reducing, or preventing amyloid β fibril formation; inhibiting neurodegeneration or cellular toxicity induced by amyloid β; inhibiting amyloid β induced inflammation; or enhancing the clearance of amyloid β from the brain.

For convenience, some definitions of terms referred to herein are set forth below.

Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque). (See, e.g., M P Lambert, et al., Proc. Nat'l Acad. Sci. USA 95, 6448-53 (1998).)

“Pharmaceutically acceptable” denotes compounds, materials, compositions, or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” includes, for example, derivatives of compounds modified by making acid or base salts thereof, as described further below and elsewhere in the present application. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid. Pharmaceutically acceptable salts may be synthesized from the parent compound which contains a basic or acidic moiety by conventional chemical methods. Generally, such salts may be prepared by reacting the free acid or base forms of these compounds with a stoichiometric amount of the appropriate base or acid in water or in an organic solvent, or in a mixture of the two.

“Inhibition” of amyloid deposition includes preventing or stopping of amyloid formation, e.g., fibrillogenesis, inhibiting or slowing down of further amyloid deposition in a subject with amyloidosis, e.g., already having amyloid deposits, and reducing or reversing amyloid fibrillogenesis or deposits in a subject with ongoing amyloidosis. Inhibition of amyloid deposition is determined relative to an untreated subject, or relative to the treated subject prior to treatment, or, e.g., determined by clinically measurable improvement in pancreatic function in a diabetic patient, or in the case of a patient with brain amyloidosis, e.g., an Alzheimer's or cerebral amyloid angiopathy patient, stabilization of cognitive function or prevention of a further decrease in cognitive function or prevention of recurrence of hemorrhagic stroke due to CAA (i.e., preventing, slowing, or stopping disease progression). Inhibition of amyloid deposition may also be monitored by determining in a subject the relative levels of amyloid-β in the brain or CSF as well as in the plasma, before and after treatment.

“Modulation” of amyloid deposition includes both inhibition, as defined above, and enhancement of amyloid deposition or fibril formation. The term “modulating” is intended, therefore, to encompass prevention or stopping of amyloid formation or accumulation, inhibition or slowing down of further amyloid aggregation in a subject with ongoing amyloidosis, e.g., already having amyloid aggregates, and reducing or reversing of amyloid aggregates in a subject with ongoing amyloidosis; and enhancing amyloid deposition, e.g., increasing the rate or amount of amyloid deposition in vivo or in vitro. Amyloid-enhancing compounds may be useful in animal models of amyloidosis, for example, to make possible the development of amyloid deposits in animals in a shorter period of time or to increase amyloid deposits over a selected period of time. Amyloid-enhancing compounds may be useful in screening assays for compounds which inhibit amyloidosis in vivo, for example, in animal models, cellular assays and in vitro assays for amyloidosis. Such compounds may be used, for example, to provide faster or more sensitive assays for compounds. In some cases, amyloid enhancing compounds may also be administered for therapeutic purposes, e.g., to enhance the deposition of amyloid in the lumen rather than the wall of cerebral blood vessels to prevent CAA. Modulation of amyloid aggregation is determined relative to an untreated subject or relative to the treated subject prior to treatment.

The term “subject” includes living organisms in which amyloidosis can occur. Examples of subjects include humans, monkeys, cows, sheep, goats, dogs, cats, mice, rats, and transgenic species thereof.

“Treatment” of a subject includes the application or administration of a composition of the invention to a subject, or application or administration of a composition of the invention to a cell or tissue from a subject, who has a amyloid-β. related disease or condition, has a symptom of such a disease or condition, or is at risk of (or susceptible to) such a disease or condition, with the purpose of curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of dementia. The treatment or amelioration of symptoms can be based on objective or subjective parameters; including the results of a physical examination or a psychiatric evaluation.

A chemical library of 100 compounds (see Table 1) was screened and a structure-based design of substituted BPEs was used to construct small molecule inhibitors against TTR-associated amyloidosis. These small molecule-based inhibitors of TTR amyloidosis were evaluated under different conditions to demonstrate kinetic stabilization independent of experimental conditions. T4, a known inhibitor and natural ligand of TTR, was used for comparison in these screening studies.

Screening of a chemical library identified inhibitors of acid-mediated TTR fibril formation. This data is shown in Table 1.

TABLE S1
Screening of a chemical library of 100 compounds (100 μM) for potential
inhibitors of WT-TTR (7.2 μM) amyloidosis at pH 4.4, 37° C.

| S. | ID | | % | % |
|---|---|---|---|---|
| N. | number | Structure | FF | Inhibition |
| 1 | 0350-0159 | 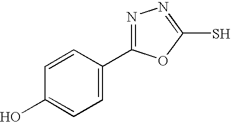 | 19.24 | 80.76 |
| 2 | 0350-0160 | 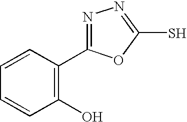 | 13.84 | 86.16 |
| 3 | 1300-0073 | 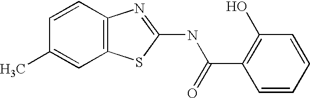 | 368.24 | 0 |
| 4 | 1544-0071 | 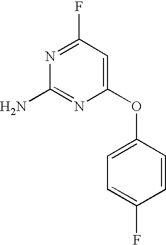 | 83.98 | 16.03 |
| 5 | 1544-0078 | 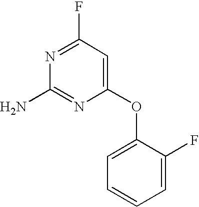 | 54.39 | 45.61 |
| 6 | 1544-0079 | 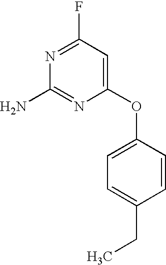 | 10.4 | 9.71 |
| 7 | 1545-0131 | 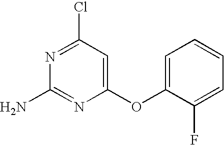 | 52.41 | 47.59 |
| 8 | 1934-0113 | 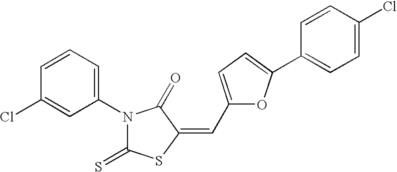 | 106.76 | 0 |
| 9 | 1989-9396 | 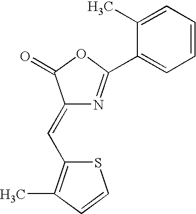 | 115.22 | 0 |
| 10 | 1996-0170 | 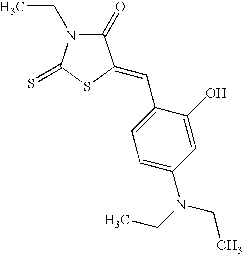 | 176.91 | 0 |
| 11 | 2019-0006 | 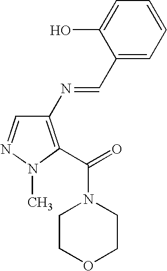 | 50 | 50 |
| 12 | 2027-0159 | 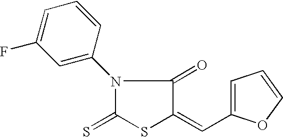 | 339.87 | 0 |
| 13 | 2027-0438 | 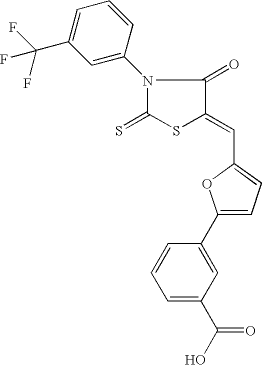 | 448.65 | 0 |
| 14 | 2189-0710 | 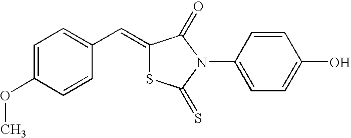 | 281.08 | 0 |
| 15 | 2189-0711 | 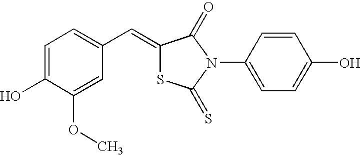 | 230.41 | 0 |
| 16 | 2189-0835 | 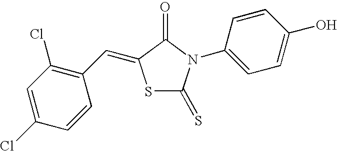 | 291.22 | 0 |
| 17 | 2279-3516 | 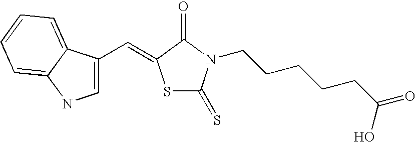 | 126.08 | 0 |
| 18 | 2369-0691 | 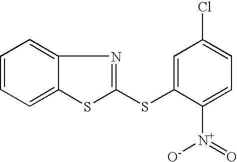 | 8.2 | 91.80 |
| 19 | 3062-0073 | 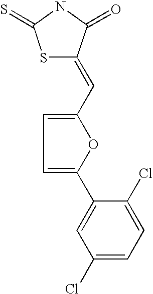 | 285.8 | 0 |
| 20 | 3062-0586 | 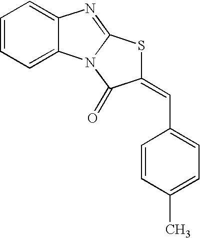 | 125 | 0 |
| 21 | 3062-0592 | 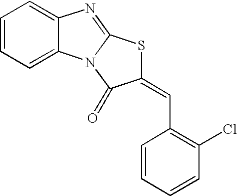 | 70.95 | 29.05 |
| 22 | 3232-0353 | 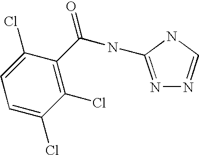 | 11.49 | 88.51 |
| 23 | 3453-1335 | 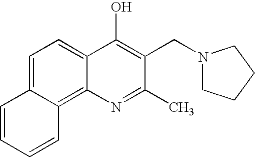 | 66.89 | 33.11 |
| 24 | 3453-1337 | 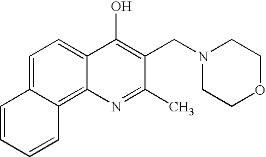 | 69.6 | 30.41 |
| 25 | 3453-1352 | 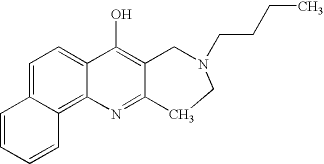 | 75.68 | 24.32 |
| 26 | 3453-1353 | 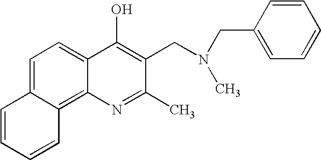 | 104.73 | 0 |
| 27 | 3615-0252 | 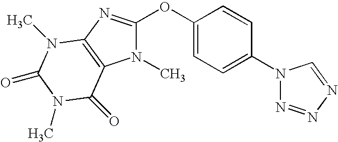 | 154.73 | 0 |
| 28 | 3232-1704 | 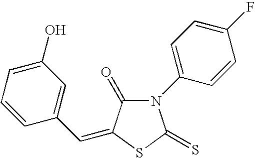 | 309.19 | 0 |
| 29 | R052-0946 | 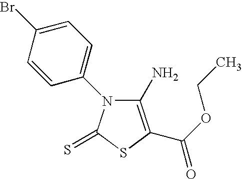 | 163.51 | 0 |
| 30 | R052-0949 | 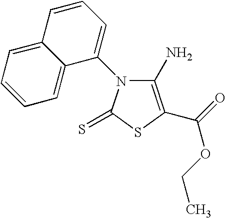 | 145.95 | 0 |
| 31 | R052-1596 | 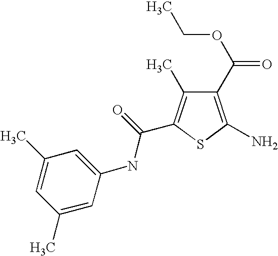 | 87.84 | 12.16 |
| 32 | R052-1597 | 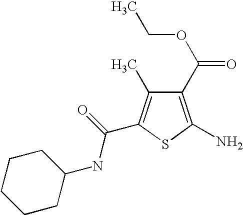 | 58.11 | 41.89 |
| 33 | R052-1661 | 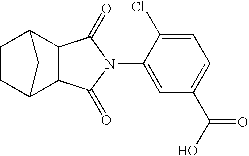 | 62.16 | 37.84 |
| 34 | R052-1661 | 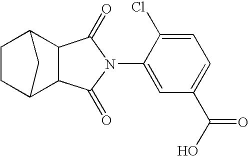 | 103.5 | 0 |
| 35 | K839-0124 | 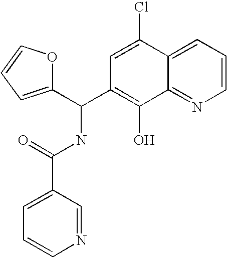 | 36.7 | 63.3 |
| 36 | K839-0116 | 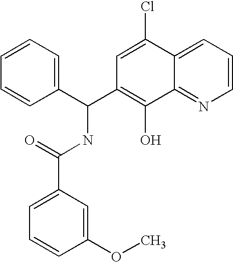 | 34.7 | 65.3 |
| 37 | K839-0117 | 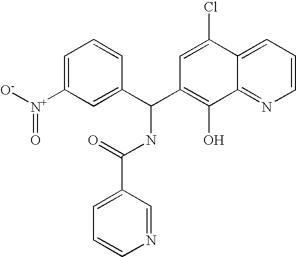 | 72.77 | 27.23 |
| 38 | K839-0113 | 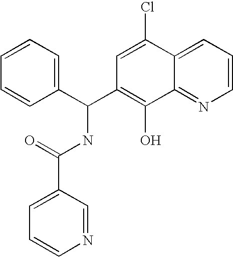 | 29.6 | 70.4 |
| 39 | K839-0067 | 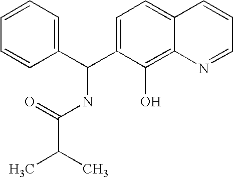 | 4.6 | 95.4 |
| 40 | K839-0017 | 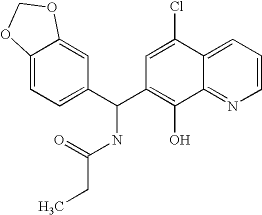 | 41.89 | 58.11 |
| 41 | K832-3346 | 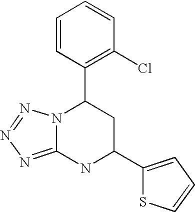 | 0.0 | 100 |
| 42 | K805-0132 | 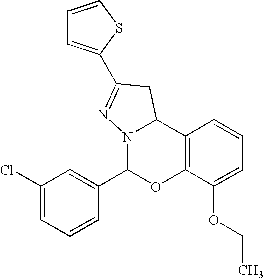 | — | 0 |
| 43 | K784-7224 | 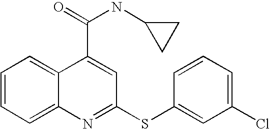 | 41.84 | 58.16 |
| 44 | K784-7215 | 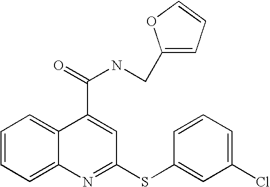 | 60.7 | 39.3 |
| 45 | K292-1247 | 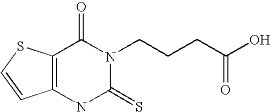 | −2.03 | 102.03 |
| 46 | K292-1247 | 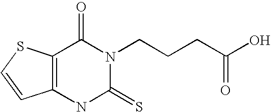 | 100.7 | 0 |
| 47 | K286-4369 | 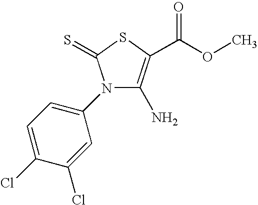 | 84.46 | 15.55 |
| 48 | K284-2958 | 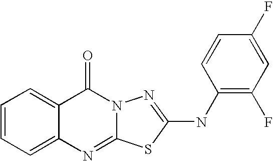 | −2.03 | 102.03 |
| 49 | K088-1892 | 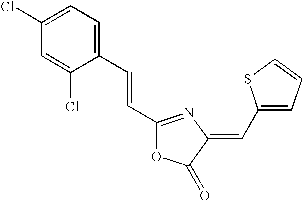 | 293.92 | 0 |
| 50 | K088-1297 | 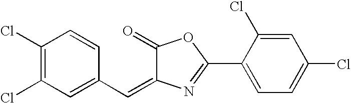 | 137.84 | 0 |
| 51 | K085-0027 | 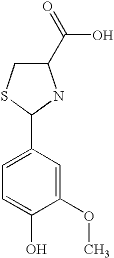 | 115.87 | 0 |
| 52 | K085-0026 | 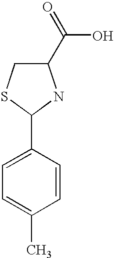 | 14.3 | 85.7 |
| 53 | K085-0025 | 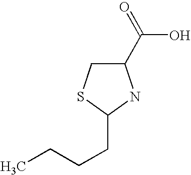 | 128.47 | 0 |
| 54 | K085-0021 | 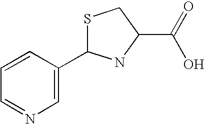 | 144.05 | 0 |
| 55 | K085-0018 | 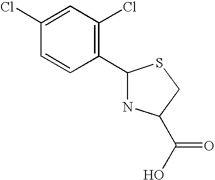 | 22.3 | 77.7 |
| 56 | K085-0014 | 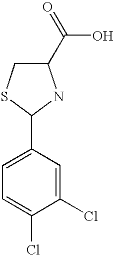 | 102.08 | 0 |
| 57 | K085-0008 | 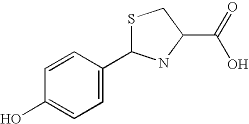 | 119.7 | 0 |
| 58 | K085-0007 | 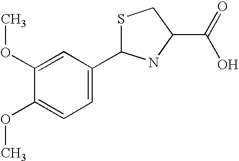 | 128.8 | 0 |
| 59 | K085-0006 | 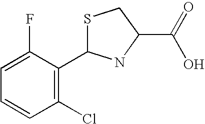 | 20.27 | 79.73 |
| 60 | K085-0004 | 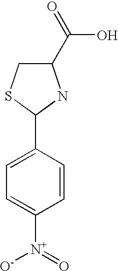 | 119.33 | 0 |
| 61 | C290-0041 | 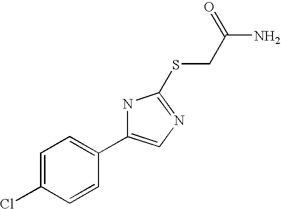 | 39.19 | 60.81 |
| 62 | K075-5819 | 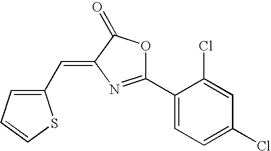 | 136.49 | 0 |
| 63 | K085-0001 | 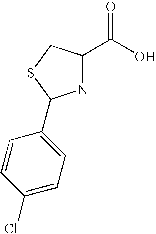 | 141.64 | 0 |
| 64 | C273-0244 | 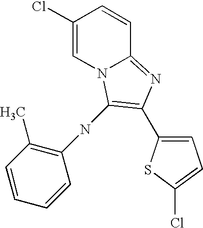 | 306.08 | 0 |
| 65 | C239-0727 | 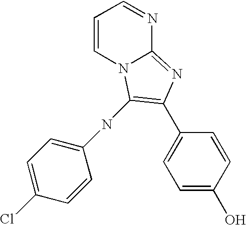 | 134.97 | 0 |
| 66 | C239-0730 | 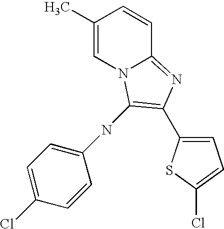 | 113.29 | 0 |
| 67 | C241-0192 | 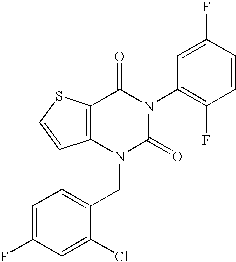 | 117.48 | 0 |
| 68 | C249-0056 | 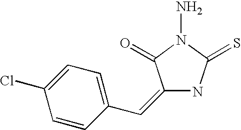 | 130.71 | 0 |
| 69 | C262-0464 | 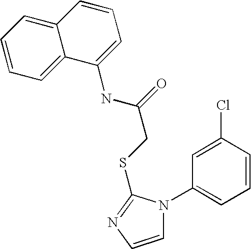 | 121.68 | 0 |
| 70 | C273-0206 | 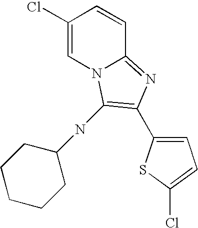 | 137.76 | 0 |
| 71 | C273-0218 | 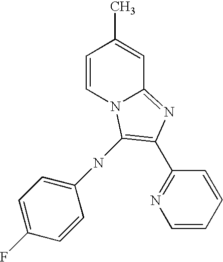 | 162.24 | 0 |
| 72 | C273-0223 | 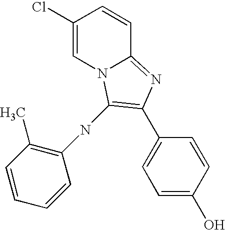 | 130.07 | 0 |
| 73 | C226-2576 | 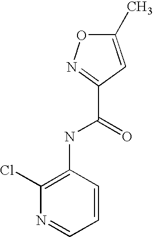 | 129.94 | 0 |
| 74 | C226-2564 | 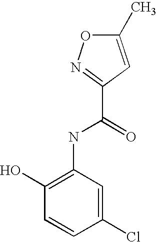 | 96.82 | 3.18 |
| 75 | C218-0295 | 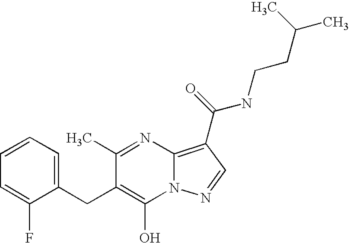 | −86.71 | 91.71 |
| 76 | C218-0299 | 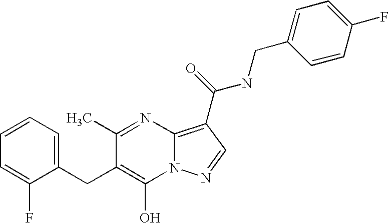 | 46.85 | 53.15 |
| 77 | C157-0040 | 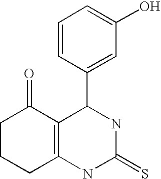 | 106.29 | 0 |
| 78 | C146-0661 | 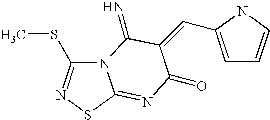 | 202.1 | 0 |
| 79 | C146-0184 | 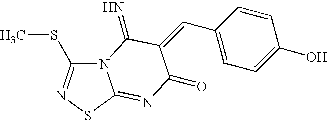 | 72.73 | 27.27 |
| 80 | C146-0181 | 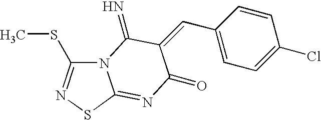 | 67.13 | 32.87 |
| 81 | C087-0783 | 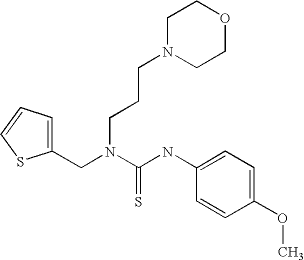 | 102.1 | 0 |
| 82 | C087-0785 | 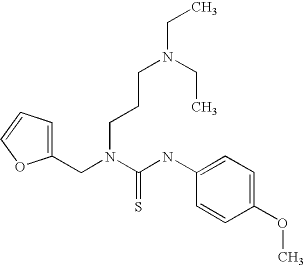 | 106.99 | 0 |
| 83 | C087-0811 | 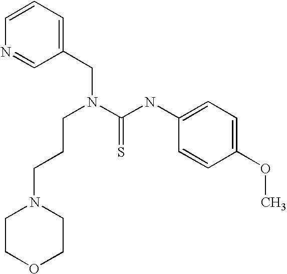 | 98.60 | 1.4 |
| 84 | C087-0823 | 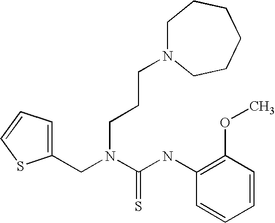 | 103.5 | 0 |
| 85 | C087-0905 | 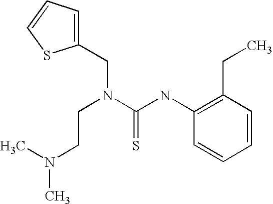 | 108.4 | 0 |
| 86 | C087-1168 | 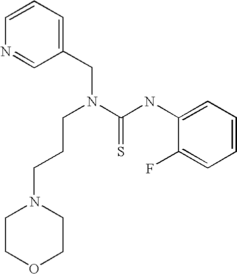 | 99.30 | 0.70 |
| 87 | C087-1176 | 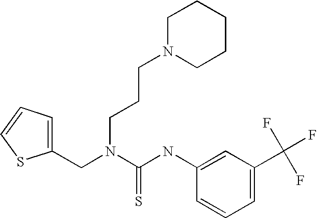 | 86.71 | 13.3 |
| 88 | C136-0215 | 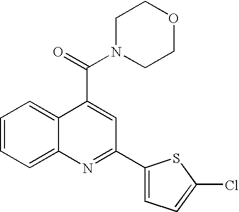 | 155.94 | 0 |
| 89 | C077-0216 | 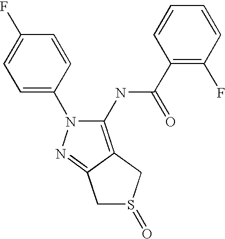 | 104.2 | 0 |
| 90 | C077-0215 | 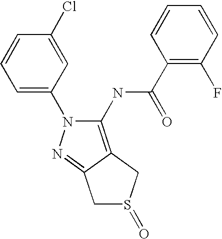 | 89.51 | 10.45 |
| 91 | C066-2523 | 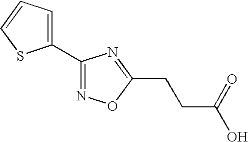 | 32.14 | 67.86 |
| 92 | C066-1627 | 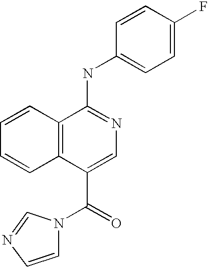 | — | 0 |
| 93 | 3996-0165 | 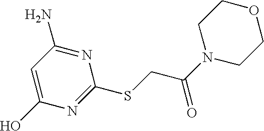 | 111.91 | 0 |
| 94 | 4358-1230 | 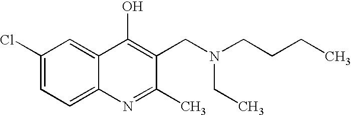 | 79.73 | 20.27 |
| 95 | 4358-1240 | 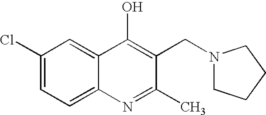 | 81.76 | 18.24 |
| 96 | 5001-0261 | 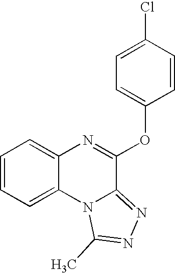 | 114.32 | 0 |
| 97 | 5594-0187 | 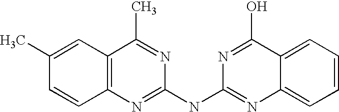 | 68.92 | 31.08 |
| 98 | 5629-0544 | 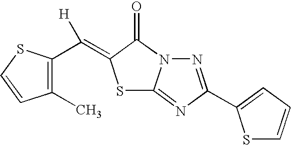 | 77.77 | 22.3 |
| 99 | 5847-1646 | 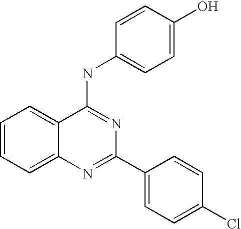 | 95.95 | 4.06 |
| 100 | 8004-9890 | 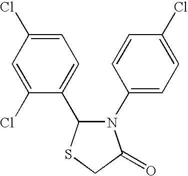 | 20.27 | 79.73 |

In addition, a series of BPE derivatives were synthesized and their ability to arrest TTR amyloidosis was tested using a variety of biochemical and biophysical methods. The derivatives disrupt preformed fibers and restore the native tetrameric state of TTR.

Compounds of particular interest are represented by the following formula:

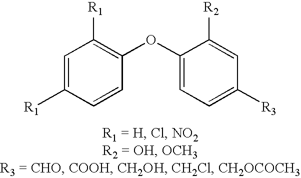

TABLE 2
Potencies of Biphenyl Ethers and Other New Structural
Templates as TTR Amyloidosis Inhibitors^{a}

| | | % FF | | IC₅₀ ± SE | Dissociation Constant (nM) | | Stoichio- | Calculated |
|---|---|---|---|---|---|---|---|---|
| S.N. | Compounds | 1:1 | 1:3 | (μM) | K_{d1} | K_{d2} | metry | energy |
| 1 | 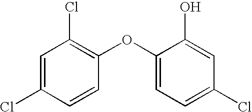 | 75 | 66 | 53.5 ± 7.01 | 144000 ± 5000 | 190.00 | 1:3.1 | −3.225 |
| 2 | 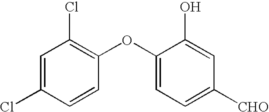 | 0 | 0 | 0.52 ± 0.01 | 13.9 ± 5.01 | 525.0 ± 60.10 | 1:1 | −3.822 |
| 3 | 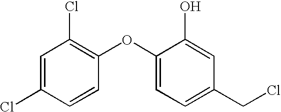 | 5 | 0 | 0.85 ± 0.08 | 5.7 ± 1.21 | 1000 ± 189 | 1:1 | −3.0592 |
| 4 | 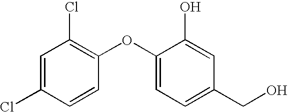 | 12 | 2.6 | 1.36 ± 0.05 | 0.3 ± 0.14 | 13800 ± 1021 | 1:1 | −3.722 |
| 5 | 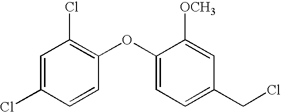 | 26 | 0 | 3.16 ± 0.04 | ND | | 1:2 | −3.567 |
| 6 | 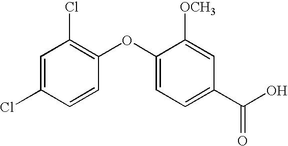 | 10 | 0 | 0.475 ± 0.03 | 0.2 ± 0.08 | 8300 ± 100 | 1:1 | −3.945 |
| 7 | 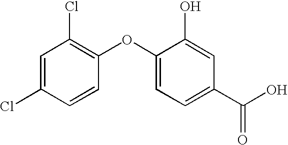 | 29 | 13.3 | 2.59 ± 0.14 | 27.0 ± 8.03 | 3.0 ± 0.78 | 1:2 | −2.723 |
| 8 | 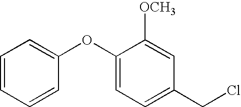 | 25 | 0 | 1.94 ± 0.09 | ND | | 1:1 | ND |
| 9 | 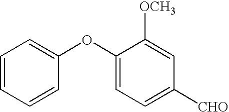 | 12 | 0 | 1.31 ± 0.08 | 88.0 ± 10.06 | * | 1:1 | −3.224 |
| 10 | 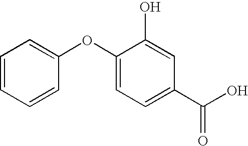 | 32 | 5 | 3.18 ± 0.16 | 27 ± 4.10 | 292.0 ± 2.81 | 1:2.4 | −2.964 |
| 11 | 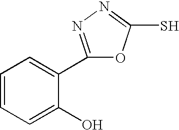 | 21 | 0 | 3.1 ± 0.05 | 42 ± 2.35 | 72.6 ± 10.32 | 1:2.4 | −3.1564 |
| 12 | 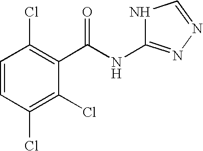 | 11 | 3.6 | 1.06 ± 0.01 | 120.0 ± 32.11 | * | 1:3.7 | −3.192 |

| | | | | | | | |
|---|---|---|---|---|---|---|---|
| 13 | 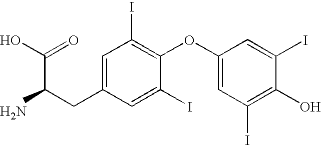 | 48 | 7 | 7.17 ± 0.21 | ND | ND | ND |

^{a}% FF: The percent fibril formation of TTR (7.2 μM). The inhibition was tested at TTR to inhibitor ratio of 1:1 and 1:3 which corresponds to 7.2 μM and 21.6 μM inhibitor concentrations, respectively, at pH 4.4, 37° C. Fibril formation by TTR in the absence of inhibitors was considered as 100%. IC₅₀ value, i.e., inhibitor concentration at which there is 50% reduction in fibril formation. Stoichiometry, the number of equivalents of inhibitor bound to one equivalent of TTR. Results presented are mean of five different experiments. ND, not determined; *, data fitted well to a single binding site.

Compounds according to this invention can be synthesized as shown in Scheme 1, Scheme 2 or by other methods.

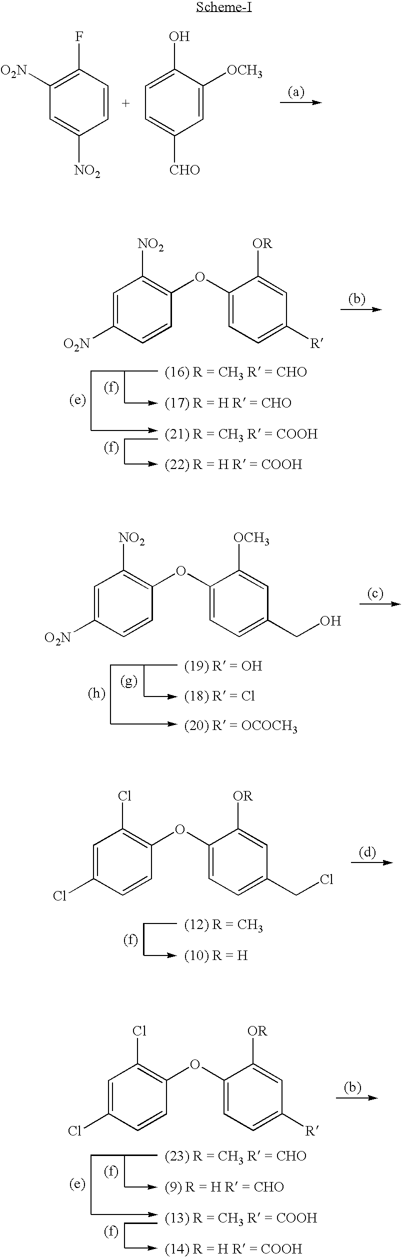

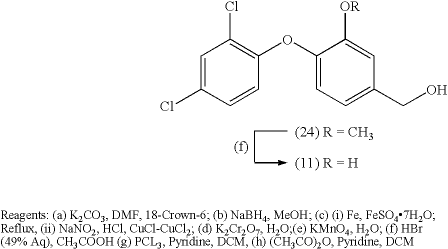

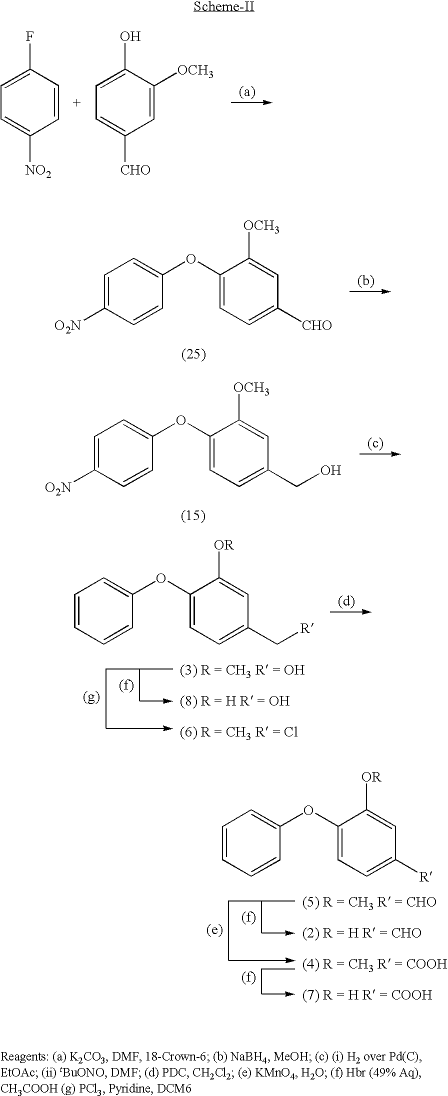

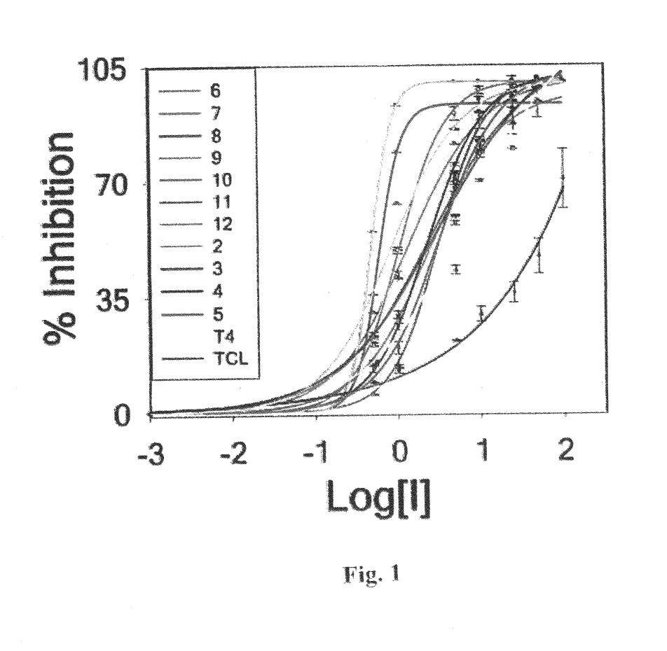

The experimental results described below and shown in FIGS. 1-10 and Table 2 demonstrate that several biphenyl ether derivatives are excellent inhibitors of TTR (7.2 μM) fibril formation. Most of them have shown ˜100% inhibition of amyloidosis at 21.6 IlM concentration when tested against 7.2 μM of TTR. The entire structure of 2, 3, and 6 appears to be important for their efficacy. Correlation of their structure with activity show that the R₁═C₁ substituted phenyl ring is essential for inhibition as can be discerned from the poor activity of R₁═NO₂ substituted BPE as shown in Table 3.

TABLE 3
Inhibition of WT-TTR (7.2 μM) amyloidosis by
designed Biphenyl Ethers (50 μM)

| S. | | | % FF at |
|---|---|---|---|
| No | Structure | IC₅₀ | 50 μM |
| 1 (1) | 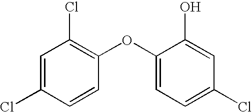 | 53.5 | 41.00 |
| 2 | 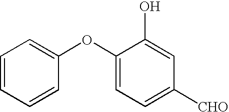 | ND | 112.24 |
| 3 | 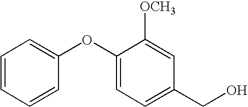 | ND | 61.19 |
| 4 | 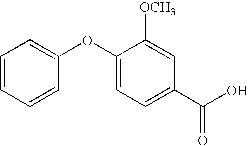 | 41.00 | 40.00 |
| 5 (9) | 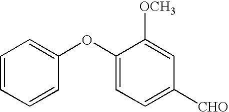 | 1.31 | 0.00 |
| 6 (8) | 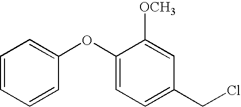 | 1.94 | 0.00 |
| 7 (10) | 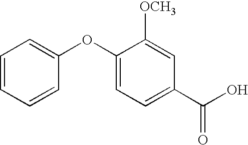 | 3.18 | 5.0 |
| 8 | 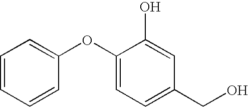 | 45.0 | 70.00 |
| 9 (2) | 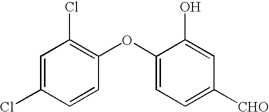 | 0.52 | 0.00 |
| 10 (3) | 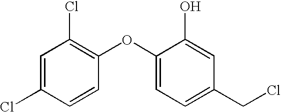 | 0.85 | 0.00 |
| 11 (4) | 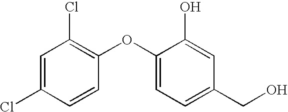 | 1.36 | 11.48 |
| 12 (5) | 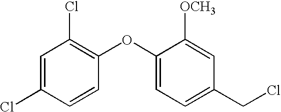 | 3.16 | 0.0 |
| 13 (6) | 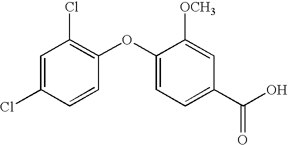 | 0.475 | 0.00 |
| 14 (7) | 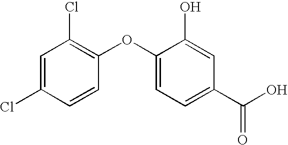 | 2.59 | 0.00 |
| 15 | 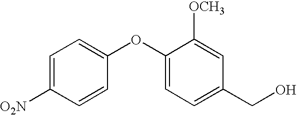 | ND | 89.51 |
| 16 | 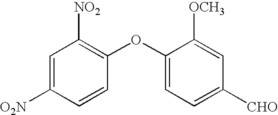 | ND | 66.00 |
| 17 | 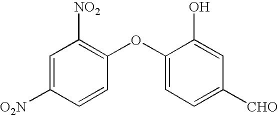 | 55.0 | 40.50 |
| 18 | 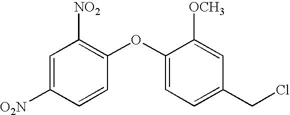 | ND | 79.00 |
| 19 | 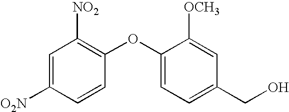 | 20.0 | 28.00 |
| 20 | 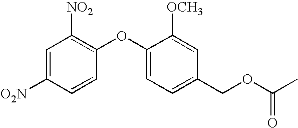 | ND | 69.00 |
| 21 | 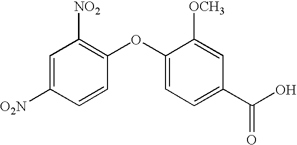 | 20 | 48.00 |
| 22 | 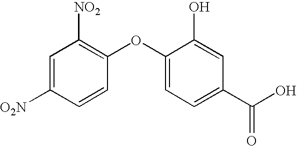 | ND | 75.00 |

The IC₅₀ value i.e. inhibitor concentration at which there is 50% reduction in fibril formation. Stoichiometry, the number of equivalents of inhibitors binds to one equivalent of TTR. Results presented are mean of 5 different experiments. ND, not determined.

Compound 10 as shown in Table 2 an analogue of 2, lacking both chlorine atoms, is less effective, indicating the importance of van der Waals interactions for their binding to TTR, a prerequisite for tetramer stabilization. Substitution at R₂═OH by OCH₃ in compound 7 decreases the activity. In contrast, activity was increased in compound 6, indicating that substitution at R₃ position is valuable. Inhibitors 2, 3, and 6, which are analogues of triclosan, also exhibited high potency signifying that carbonyl, carboxylic, and alkyl halide substitutions at R₃ position improve their efficacy over triclosan. Thus, the Cl group at the R₁ position and the carbonyl group at the R₃ position of these biphenyl derivatives are important for inhibition of TTR The number of active R₂- and R₃-substituted BPEs suggest that additional manipulation of BPE at these positions could yield inhibitors that are even more potent.

Of the 100 compounds from the chemical library, 41 compounds inhibited TTR fibril formation. Of these, two compounds (11 and 12) shown in Table 2 were found to be the most potent. The compounds thus identified from the chemical library are heterocyclic compounds. The high potency and the significant differences in their molecular scaffolds as compared to the existing core structures (biphenyl ether, biphenyl amine, biphenyl, etc.), makes them useful for the design of potent TTR-specific amyloidosis inhibitors.

Fibril Formation Assay. The stagnant fibril formation assay at pH 4.4 showed that all the synthetic BPEs examined inhibited TTR amyloidosis at stoichiometric concentration (Table 3). Out of 24 designed BPEs, nine compounds (represented as 2-10 in Table 2) inhibited the process >90% when 3 molar equivalent of them were used over TTR (BPEs=21.6 μM, TTR=7.2 μM), except triclosan (˜24%), and were therefore selected for a detailed study. BPEs were found more effective as inhibitors than T4.

Fibrillization of TTR with added compounds 1-12 buffered at pH 4.4 was similar, suggesting the affinity of inhibitors for TTR is greater than the propensity of TTR tetramer dissociation and misfolding.

Out of the 100 compounds from the chemical library, 41 compounds were found to inhibit fibril formation at 100 μM concentrations (Table 1). Of these, only two compounds 11 and 12 were found to be the most potent, as they inhibited TTR (7.2 μM) fibrillization completely at 21.6 μM (Table 2). IC₅₀ for compounds 2-12 are in the range of 0.47-3.5 μM, which are lower than IC₅₀ of 7.1 μM for its natural ligand T4 (Table 2). The dose-dependent curves for inhibition of TTR amyloid formation by these compounds are shown in FIG. 1. The experiment was performed at 37° C. The data were fitted in to sigmoid equation as given in, and the IC₅₀ value was calculated graphically. Results are the mean of five independent experiments with error bar.

The dissociation constant K_{d1} of compounds 2-12 at pH 4.4, are in nanomolar range compared to 144.0 μM of triclosan (Table 2). For compounds 2, 3, 4, 6, and 10, the K_{d2} is 37-10000-fold higher than their respective K_{d1}, suggesting negative cooperativity for their binding to the second site of TTR.

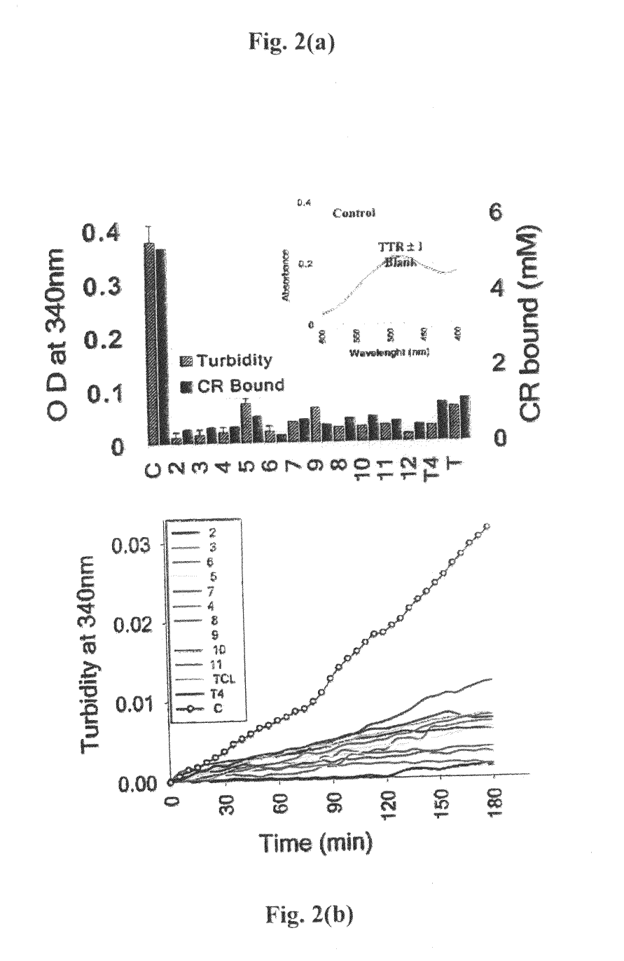

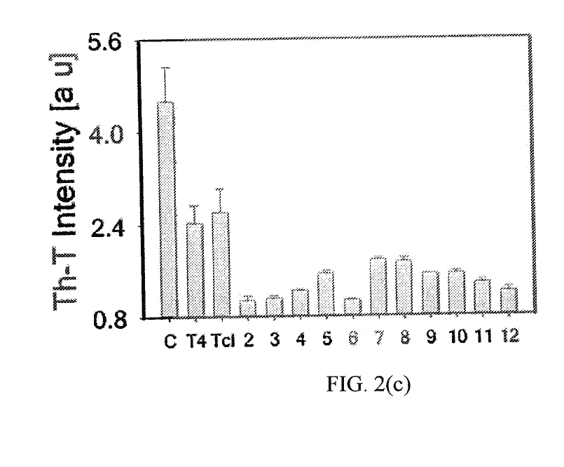

Compounds buffered at pH 4.4 have also acted as potent inhibitors of the process. Table 2 shows the stoichiometry of binding of the compounds 1-12 to TTR tetramer. Further, in order to rule out the presence of any fibrillar species, inhibition of fibril formation by compounds 1-12 was tested by Congo red binding. A comparison of the results of turbidity assay at 340 nm and of quantitative Congo red binding to fibril formed by TTR in the presence and absence of inhibitors is shown in FIG. 2a which is a comparative bar diagram of turbidity (shaded bars) and quantitative Congo red binding (black bars) assay to quantitate TTR (7.2 μM) fibrillogenesis in the absence and presence of 3 equiv (21.6 μM) of compounds 1-12 and T4. The results shown are means of 3-5 different observations. Congo red binding to the TTR in the presence and absence of inhibitors after 15 days of incubation at pH 4.4 was monitored by scanning for absorption from 400-600 nm (spectra in the inset). Experiments were carried out at 37° C.

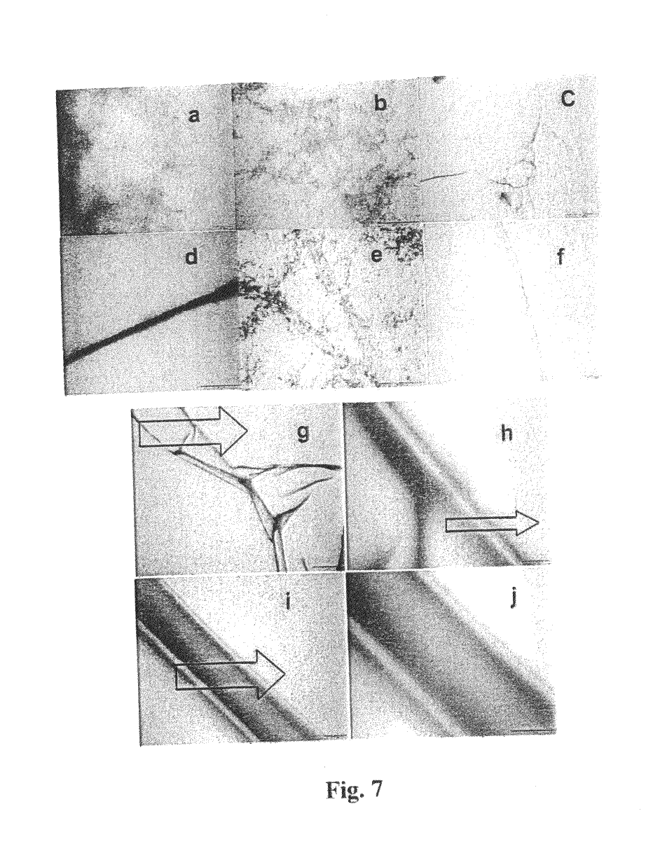

While unligated TTR exhibited maximum binding of Congo red, it was significantly reduced in the presence of compounds 1-12. Even 7-30 days old samples in the presence of compounds 1-12 exhibited diminished binding of Congo red to TTR. TTR fibrillization in the presence and absence of compounds 1-12 was further examined by Transmission electron micrography (TEM). As is evident from TEM pictures (FIGS. 7(a) to (j)) the samples containing compounds 2-12 were devoid of any fiber or amorphous aggregates. FIG. 7(a) to (j) shows the Transmission electron micrographs of TTR fibers. TTR fiber formed under acidic condition FIG. 7(a) shows an undiluted sample, 1:10 diluted sample is shown in FIGS. 7(b) and 1:100 diluted is shown in FIG. 7(c), FIG. 7(d) shows Twisted tangled protofibril FIG. 7(e) mature fiber, FIG. 7(f) shows mature fiber along with aggregates in 1 month old sample; FIG. 7(g) shows tangled branched nano tubes like structure. FIG. 7(h) shows a part of nanotube magnified in FIG. 7(i & j). In contrast, BPE-untreated samples exhibit fiber and fibrillar aggregates in abundance.

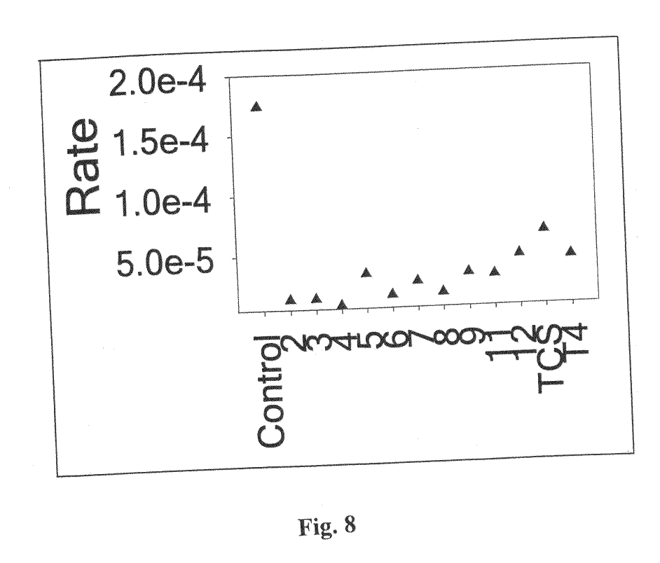

Kinetics of Fibril Formation. The kinetics of fibril formation in the presence and absence of the compounds were monitored by linear increase in the turbidity at 340 nm for 3 h. The increase is prominent in the absence of inhibitors, while it was significantly reduced in the presence of the inhibitors and T4. The inhibition was >80% by compounds 2-12 and 62% by triclosan as compared to 75% inhibition by T4 (FIG. 2b). FIG. 2(b) shows the time course of TTR (7.2 μM) fibril formation in the presence and absence of compounds 1-12 and T4 (21.6 μM) at 37° C., pH 4.4. Turbidity at 340 nm was monitored for 3 h and is plotted against time. The results are the mean of three different experiments done in triplicate. Standard error was <10%. The plot shows the rate of fibril formation in the presence and absence of compounds 1-12 and T4 in preventing TTR fibril formation is given in FIG. 8: The plot shows the rate of fibril formation in the presence and absence of compounds 1-12 and T4 in preventing TTR fibril formation. The kinetics of inhibitions was studied by taking 7.2 μM TTR and 21.6 of inhibitors in a 96 microwell plate. The change in turbidity was monitored at 340 nm in the microplate reading using Magellan software. Change in absorbance at 340 nm was plotted against time in the presence and absence of inhibitors. Initial rate of fibril formation was calculated from the value of slope by linear fitting of the change in absorbance at 340 nm as a function of time. These results are consistent with the results of stagnant fibril formation assay (FIG. 2a).

The IC₅₀ determined for compounds 2, 3, and 6 are lowest values observed so far for the inhibition of TTR fibrillization. The low IC₅₀ values (less than 1 equiv of TTR) for all these ligands clearly indicate that substantial inhibition of amyloidogenesis does not require kinetic stabilization of every TTR tetramer by the binding of a small molecule. Misfolded monomeric TTR aggregates into amyloid fibrils via a straightforward self-association mechanism where all forward steps in the pathway are favorable, whereas the rate of aggregation is dependent on the concentration of the misfolded TTR monomer. (Hurshman, et al. Biochemistry 2004, 43,7365-7381). Since the concentration of monomer depends on dissociation of the tetramer, controlling the energetics of tetramer dissociation will allow significant control over the amyloidogenic monomer concentration dictating the rate of TTR amyloidogenesis. Thus, tight binding of a small inhibitory molecule at substoichiometric concentrations (less than 1 equiv) may be sufficient to reduce the concentration of amyloidogenic monomeric TTR in the serum, thereby preventing the disease. This was evident by the kinetics of fibril formation in the presence and absence of inhibitors. A significant decrease in the rate of fibril formation by compounds 2-12 compared to the control was observed. The time course of TTR fibrillogenesis shows that it lacks a lag phase and is not seedable, which is in agreement with earlier reports.

The dissociation constant for all these compounds at pH 4.4 correlates well with their efficacy. The strong negatively cooperative binding of compounds 2, 3, 4, 6, and 10 suggests that the binding of ligand to one site is sufficient to stabilize the tetramer to prevent amyloidosis. K_{d}s are low enough in case of 7, 9, 11, and 12 to saturate both binding sites in TTR at 3.6 μM, i.e., equivalent to the physiological concentration (3.6 μM), ensuring that there is enough inhibitor to saturate both the binding sites. These biphenyl ether derivatives are better inhibitors as compared to T4 at lower concentrations. The inhibition of fibrillogenesis by compounds were further evaluated by Congo red binding and Thioflavin-T fluorescence (FIGS. 2a and 2c), as both bind specifically with protein fibers. Significant decrease in the amount of Congo red binding in the presence of inhibitors indicates absence of fibril formation compared to the unligated TTR. Similarly, binding of Th-T to TTR fibril and thus fluorescence was also decreased in the presence of compounds 1-12, providing further supporting the inhibition of TTR amyloidosis by these compounds.

Thioflavin-T (Th-T) Fluorescence. Th-T fluorescence was monitored in the presence and absence of the inhibitors at pH 4.4 at 0 and 72 h. The intensity of Th-T was high in the absence of the compounds 2-12 and significantly less in their presence (FIG. 2c) as both bind specifically with protein fibers.

FIG. 2(c) shows thioflavin fluorescence after binding to TTR (7.211M) in the presence and absence of compounds 1-12 and T4 (21.6 11M) under fibrillization condition Fibrils were produced by acidification at pH 4.4 for 7 h at 37° C. and monitored by the Th-T fluorescence assay. The samples were incubated with 50 μM of Th-T for 15 min at 37° C. and excited at 450 nm, and Th-T fluorescence emissions were monitored at 480 nm. Results are means of three independent experiments done in duplicate. There was no significant increase in Th-T binding even in the samples incubated for 7-30 days in the presence of compounds 1-12 (data not shown).

Significant decrease in the amount of Congo red binding in the presence of inhibitors indicates absence of fibril formation compared to the unligated TTR. Similarly, binding of Th-T to TTR fibril and thus fluorescence was also decreased in the presence of compounds 1-12, providing further supporting the inhibition of TTR amyloidosis by these compounds. Fibrils were produced by acidification at pH 4.4 for 72 h at 37° C. and monitored by the Th-T fluorescence assay.

To understand the conformational states of the TTR in the presence and absence of inhibitors, intrinsic tryptophan fluorescence of the protein was monitored.

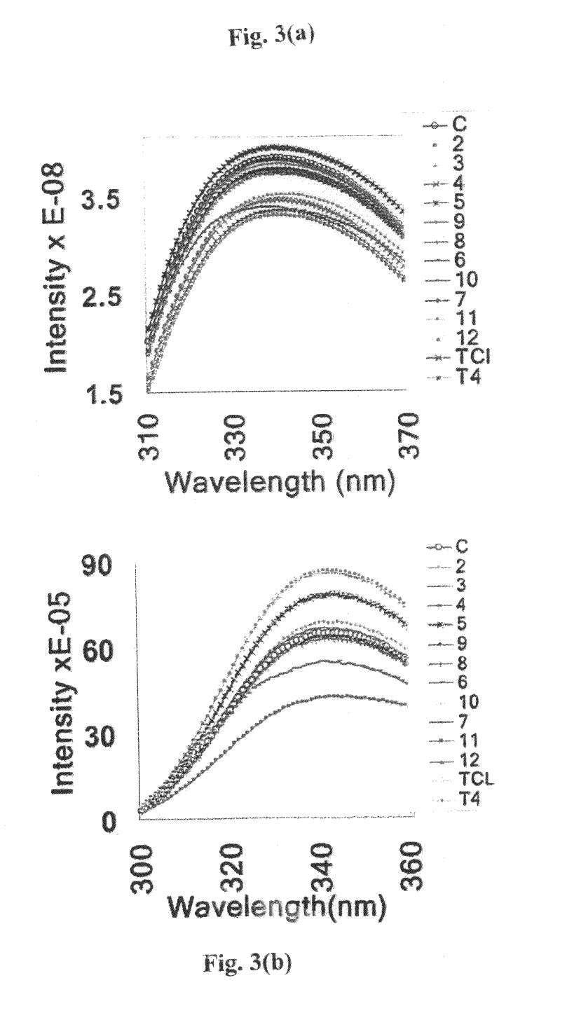

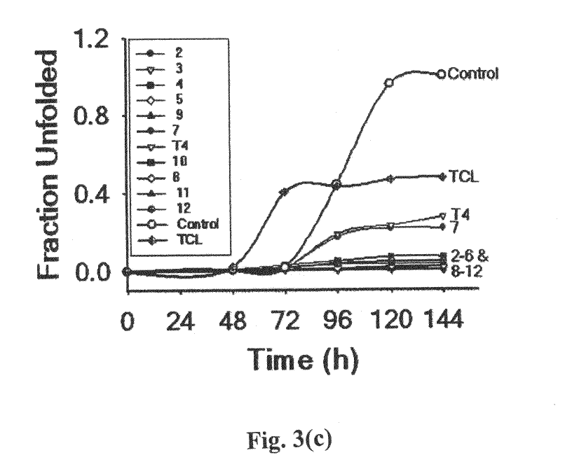

Tryptophan Fluorescence. TTR exhibits fluorescence emission maxima at 339 and 343 nm, respectively, at pH 7.2 and 4.4, indicating the presence of tryptophan residues exposed to solvent at the surface of the protein. Conformational changes in TTR induced upon binding of inhibitors therefore were studied by monitoring the intrinsic fluorescence of the protein in the presence and absence of the compounds 2-12, triclosan, and T4 both at pH 7.2 and 4.4. FIG. 3(a) and FIG. 3(b) show the conformational change in TTR induced by binding of compounds 1-12 and T4 TTR (3.6 μM) incubated in the presence and absence of compounds 1-12 (7.2 μM) at 25° C. FIG. 3 (a) shows the changes at pH 7.2, and FIG. 3(b) at pH 4.4. Change in conformation was inferred by monitoring the intrinsic fluorescence of TTR. Samples were excited at 290 nm, and emission was monitored in the range of 310-370 nm. Results are mean of three different experiments.

A significant reduction in the fluorescence intensity of TTR was observed in the presence of compounds 1-4 and 6-12 at pH 7.2 (FIG. 3a) which was more pronounced at pH 4.4 (FIG. 3b). This indicates a subtle change in the exposure of tryptophan residue(s) in TTR upon the binding of compounds 1-12, which in turn appears to be a reflection of a change of its quaternary structure. A comparison of the change in fluorescence intensity of TTR treated with the compounds 1-12 show a greater reduction at pH 4.4 as compared to that at pH 7.2, indicating that at acidic pH a greater proportion of TTR is being driven to tetramer formation by the binding of these compounds.

Reduction in intensity at pH 7.2 in the presence of compounds 1-12 indicates interaction of these compounds leading to conformational change in protein (FIG. 3a). In contrast, drastic reduction in the intensity was observed in TTR at pH 4.4 (FIG. 3b). The emission maxima of TTR under fibrillogenesis conditions (pH 4.4) showed 3 nm red shift (343 nm) compared to TTR at pH 7.2, where emission maxima was at 340 nm, signifying a subtle conformational change at pH 4.4. In the presence of inhibitors, however, decreased quenching of TTR fluorescence was observed under denaturing conditions, implying pH-independent stabilization of the protein in the presence of compounds 1-12.

Urea Denaturation. It has been reported earlier that the dissociation of the tetramer is a critical and rate-determining step in TTR related fibrillogenesis. (Jiang, X.; Buxbaum, J N.; Kelly, J. W. The V InI cardiomyopathy variant of trans thy ret in increases the velocity of rate-limiting tetramer dissociation, resulting in accelerated amyloidosis Proc. Natl. Acad. Sci. U.S.A. 2001, 98, 14943-14948). The ability of these inhibitors to impose kinetic stability on tetrameric TTR can be best evaluated by the rate of tetramer dissociation. Tetramer dissociation leads to the unfolding of the monomer in the presence of 6.0 M urea at 25° C. To understand the mechanism of inhibition of fibril formation, denaturation kinetics of TTR in 6.0 μM urea was carried out. The TTR tetramer does not denature in urea; however, dissociation to monomer is required for urea-induced tertiary structural changes which can be detected by monitoring the changes in intrinsic tryptophan fluorescence. (Hammarstrom, P. et al., Proc. Natl. Acad. Sci. U.S.A. 2002, 99, 16427-16432 and Hammarstrom P. et al. Science 2001, 293, 2459).

As the rate of TTR fibrillization is proportional to the rate of tetramer dissociation, the rate and extent after tetramer (1.8 μM) dissociation in 6.0 μM urea was monitored by evaluating the intrinsic fluorescence of TTR. Compounds 1-12 exert substantial effect on the amplitude of TTR tetramer dissociation (FIG. 3c) FIG. 3c shows the time dependence of tetramer dissociation (fraction unfolded) of TTR in the presence and absence of inhibitors in 6 M urea at 25° C. TTR (1.8 μM) incubated for 1 h with 5.4 μM of compounds 1-12 or T4 at 25° C. at pH 7.2 was used for these studies. Urea was then added to these solutions to a final concentration of 6 μM, and the unfolding of TTR was monitored by following the intrinsic fluorescence of the protein at 339 nm up to 144 h. Samples were excited at 290 nm. Fraction unfolded at each time point was plotted against time. Results are mean of three different experiments performed in duplicate. For example at 1:3 ratio of TTR to triclosan, 47% of the protein dissociates as compared to the untreated control under identical conditions. In comparison, compound 7 and T4-treated TTR under similar situation exhibits 21 and 27% dissociation, respectively. Interestingly, in the presence of compounds 2-6 and 8-12, less than 5% of the protein dissociates and unfolds even after 144 h, implying an overwhelming stabilization of TTR tetramer by these compounds (FIG. 3c).

Although not being bound by an specific theory, it is possible to infer from these data that these compounds act by kinetic stabilization of TTR tetramer under denaturing conditions over the amyloidogenic monomer, a finding consistent with the rate of fibril formation at 37° C., pH 4.4 (FIG. 2a).

The TTR tetramer dissociation is drastically slowed down in the presence of compounds 1-12, compared to the control. However, the decrease was less for T4 and triclosan, clearly indicating that the efficacies of the compounds 2-12 in increasing the energy barrier for tetramer dissociation is responsible for the stabilization of the tetramer of TTR.

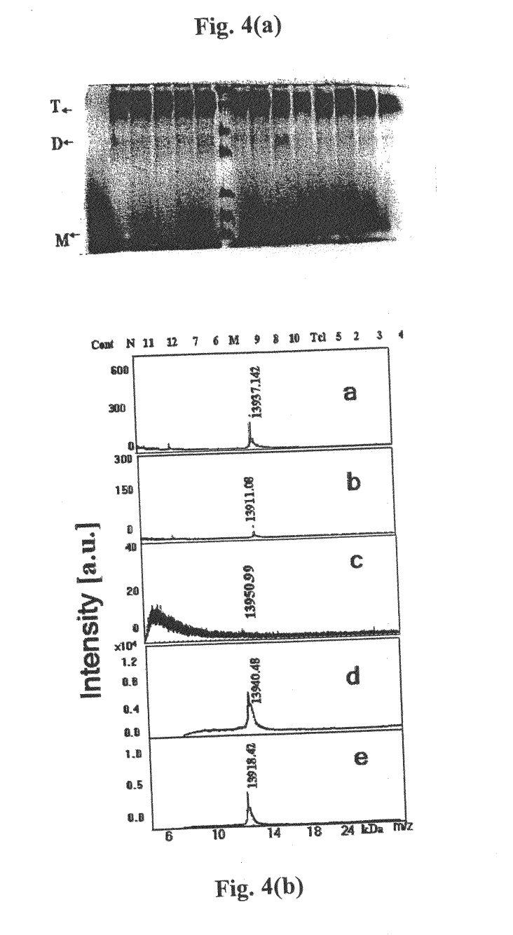

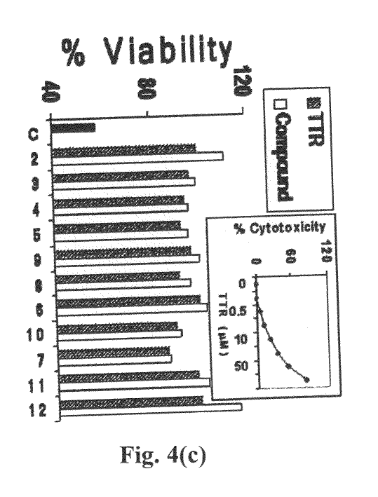

Glutaraldehyde Cross-Linking. Cross-linking experiments with glutaraldehyde clearly show that even under the conditions conducive for fibrillization, TTR exists mostly as a tetramer In the presence of compounds 1-12 and T4 (FIG. 4a). FIG. 4 (a) shows oligomeric status of TTR in the presence and absence of compounds 1-12 at pH 4.4 after 15 days of incubation under fibrillization conditions.

To understand the mechanism of inhibition of fibril formation by all the above compounds, the quaternary structure of TTR in the presence and absence of these compounds at acidic condition was characterized by glutaraldehyde cross-linking followed by the analysis of the cross-linked products by SDS-PAGE. TTR at pH 7.2 migrated predominantly as a tetramer (55 kDa), whereas at pH 4.4 it migrated as a monomer (FIG. 4a). The reaction mixtures were neutralized and the samples crosslinked with gluteraldehyde. They were then elecro-phoresed on 12% SDS-PAGE. TTR complexes with compounds 1-12 migrated mainly as the tetramer. N, is TTR at −20° C. M, molecular wt standards

However, TTR migrates predominantly as a tetramer in the presence of compounds 1-12. Only a small amount of dimer was observed in case of native TTR and TTR in the presence of compounds 1-12. In the absence of these inhibitors, the amount of tetramer was decreased with time and became zero after 3 days (data not shown). In contrast, there was no significant difference in the tetramer concentration in the presence of these inhibitors during 72 h to 30 days.

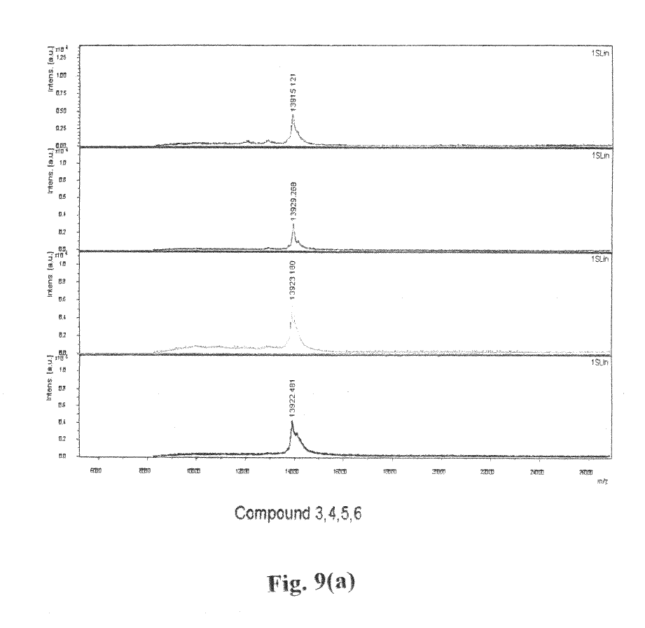

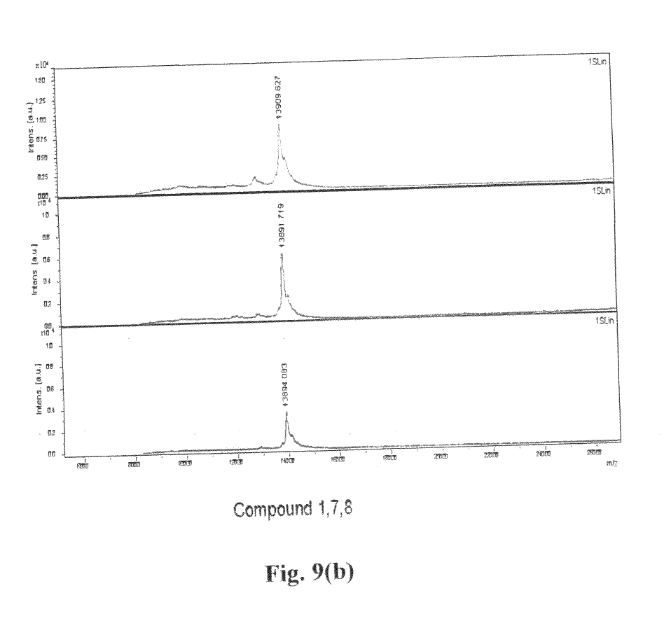

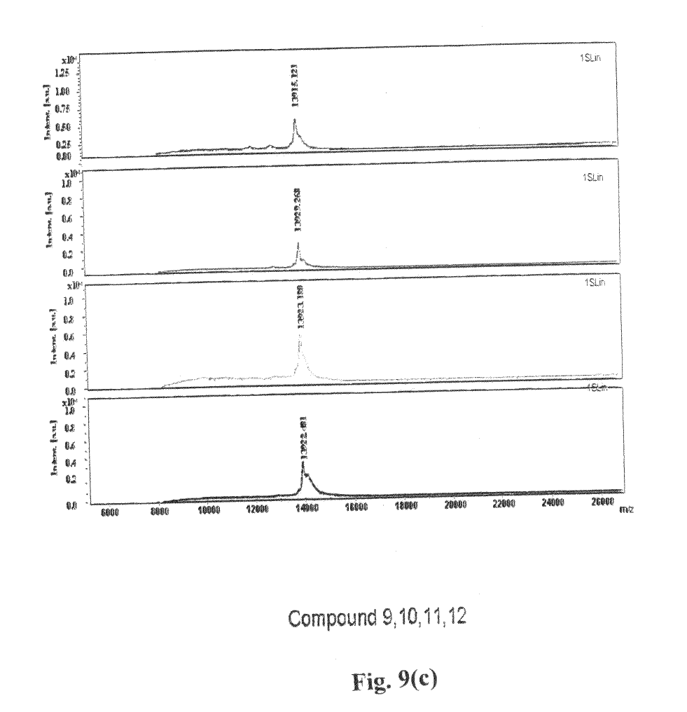

Mass Spectrometry. All the above observation clearly show that compounds 1-12 prevent fibril formation by stabilizing TTR tetramer. It can, therefore, be assumed that soluble TTR present in the reaction mixture mostly exists as a tetramer. To confirm these findings, MALDI-TOF of TTR samples treated with these inhibitors for 3, 7 and 15 day subsequent to centrifugation and filtration through 80 kDa cutoff membranes was carried out as described in the Experimental Section. As shown in FIG. 4b, (a-e) the intensity of the monomer peak gradually decreases which completely disappear by day 7 in the absence of the inhibitors (FIG. 4 (b), (a-e)). In contrast, a peak corresponding to ˜13850 Da can be observed in the presence of inhibitor 2, shown here as a representative example, even after 15-30 days of fibrillogenesis (FIG. 4(b) (c), (d) and (e)) FIG. 4 (b) shows stability of TTR treated with compounds 1-12. Mass spectra of TTR at (a) 0 h, (b) 72 h, (c) 7 days at pH 4.4 (d) native TTR and (e) in presence of inhibitor 2 after 7 days. The mass spectra of TTR with compounds complexed with inhibitors 1-2 at 15 days are given in FIG. 9. The TTR (7.2 μM) was incubated with compound 1-12 at 37° C. for 15 days under acidic condition (pH 4.4). The 15 days old samples were centrifuged to remove any fibril formed. 1 μL of the supernatant solution was mixed with 1 μL of saturated solution of sinapinic acid (3-(4-hydroxy-3,5-dimethoxy-phenyl)prop-2-enoic acid) in ethanol containing 0.5% trifluoroacetic acid. This mixture was loaded on to the target plate. Mass was recorded using Bucker MALDI-TOF instrument As shown in FIG. 9(b) in the presence of compound 1-12 TTR mainly exist as tetramer compound to unligated TTR.

In contrast, no tetramer was observed in unligated TTR, ruling out the possibility of the formation of the prefibrillar cytotoxic species in the presence of compounds 2-12 during fibril formation. The stability of TTR-inhibitor complex was determined under denaturing condition by extending the incubation time to 7-15 days. An analysis of MALDI-TOF data shows remarkable stability of TTR by BPEs compared to that observed in the presence of T4 (FIG. 4b).

Even after 30 days of incubation, TTR retains its tetrameric structure in the presence of 3 equiv of compounds 2-12 under conditions, in which the absence of these compounds would have led to fibrilization within 72 h.

Inhibition of TTR-Induced Cell Cytotoxicity by Compounds 2-12. Neuro 2A cell culture was used to detect the presence of any soluble toxic aggregates in the reaction mixture used for studying the inhibition of TTR fibrillogenesis. Preliminary experiments showed the mature TTR amyloid fibers are not cytotoxic (data not shown). However, TTR solution left under the conditions of fibrillogenesis induces toxicity to Neuro2A cells in a concentration-dependent manner in 72 h (FIG. 4(c)). FIG. 4(c) shows the TTR-induced ˜41% cytotoxicity under the fibrillogenesis conditions. FIG. 4(c) shows the efficacies of compounds 2-12 in protecting Neuro 2 cells against cytotoxicity of TTR. The graph shows the % viability of cells treated with TTR (25 μM) alone (black bar), at 2 equivalent of compound 2-12 (50 μM) with respect to TTR concentration (hatched bars), and compounds alone (open bars). Dose-dependent curve of TTR-induced cytotoxicity to Neuro2a cells is given in the inset. Results are mean of three different experiments conducted in triplicate. However, preincubation of TTR (25 μM) with the compounds 2-12 (50 μM) exhibited viability in the range of 86-99.5%. Compounds 2-12 at 50 μM by themselves had no effect on the survival of Neuro 2A cells in culture, demonstrating their nontoxic nature at the concentration used (FIG. 4(c).

The cell cytotoxicity assay shows that TTR-induced cytotoxicity was inhibited by the compounds 2-12 that stabilize the TTR tetramer (FIG. 4c). Interestingly, these inhibitors are not cytotoxic at the concentration tested and add to the potential of these BPEs as inhibitors of fibril formation and the consequent pathogenesis of the disease.

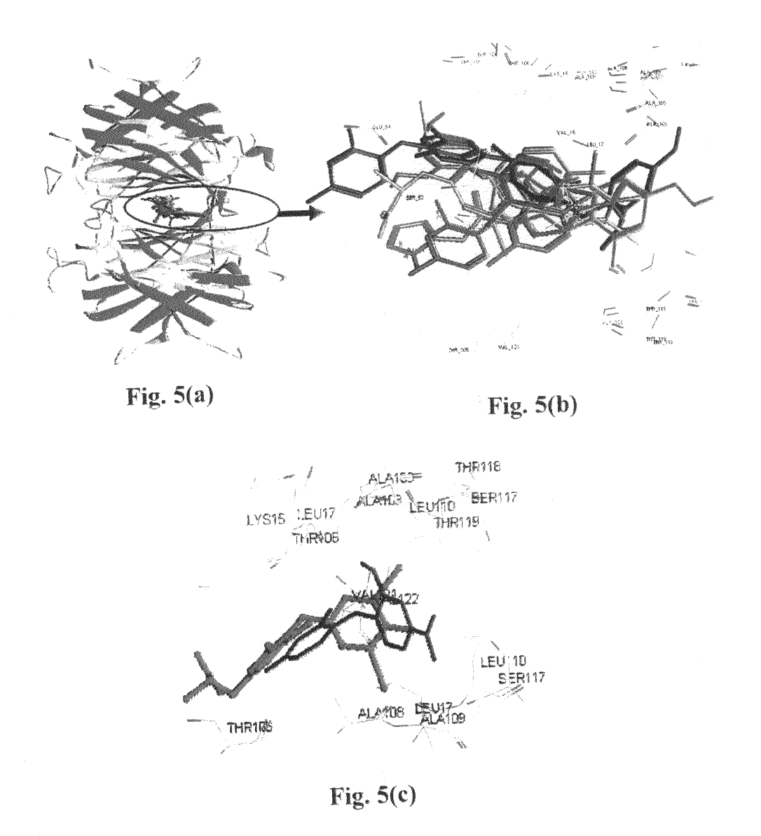

Molecular Docking. To validate and under the basis of the Inhibitory activities of these closely related BPEs in TTR amyloidosis, docking studies were performed. The crystallographic structure of the TTR-T4 complex (Wojtczak, A. Cody, V., Luft, J. R.; Pangborn, W. Structures of human transthyretic complexed with thyroxine at 2.0 resolution and 3′5′-dimitro-N-acetyl-L-thyronine at 2.2. A resolution, Acta Oystallogr, Sect. D. Biol. Crystallogr. 1996, 52, 758-765) showed its hormone-binding sites, which are composed of three symmetry-related hydrophobic small depressions, termed halogen-binding pockets. The molecular docking of all these inhibitors with TTR shows their binding to the T4 binding site with binding of one ring to P3 and the other to P1 pocket. Interactions with the residues lining the pocket are mainly through hydrophobic and electrostatic interactions. FIG. 5 shows the overlap of several of the MOE-docked BPE's and compound 11 and 12 in the thyroxine binding pocket of TTR complexed with T4. Triclosan and other biphenyl ethers with R₁=Cl bind deep in the P3 pocket of TTR while the dihalogenated phenyl ring is positioned in the outer P1 pocket of the binding cavity, viz. the two chlorine atoms of the B-ring of tricolsan are accommodated in P1 and P1′ pockets, respectively (FIG. 5). Van der Waals interactions between both the chloride substituent of the ligand and the side chains of Leu 17, Ala 108, Thr 11, and Val 121 from the adjacent TTR subunit contribute to the stabilization of the tetramer. The atoms of BPE's that are involved in extensive hydrophobic interactions with Ala 108, Leu 110, Serl 17, Thr 118, and Thr 119 of the monomer AA′ and CC′ appear to contribute significantly to the overall stability of the tetrameric structure. Thus two molecules of these BPE's bind to the two hormone-binding pockets in an antiparallel orientation. In case of R₁═H, the unsubstituted ring binds to P3 pocket and R₂ and R₃ substituted ring binds to the outer pocket stabilize the structure (FIG. 5). In the case of compounds 11 and 12, the benzene ring binds deep in the P3 pocket making hydrophobic interactions, while heteroatoms N and O of the outer ring are involved mainly in salt bridges with the residues lining the P1 pocket (FIG. 5).

An analysis of TTR-ligand docking studies indicates that the BPEs establish optimum interactions within the hormone binding site (FIG. 5) FIG. 5 shows the results of the docking of compounds 2-12 on the TTR tetramer. Molecular docking with the TTR tetramer was done with each of the inhibitors using MOE-2005 software. There are two symmetrically equivalent positions for ligands in each tetramer of TTR. For the sake of clarity, only one of the symmetry equivalent positions of the binding site for the T4 together with other inhibitors docked therein is shown (a) The overlap structure of docked compounds 2-12 to TTR and thyroxine (orange) to TTR (PDB code 2ROX). (b) Blown up version of the thyroxine binding site along with compounds 2-6, 10, and 12 overlapped with T4. Color does are T4 (orange), 2 (cyan), 3 (red), 4 (light green), 5 (blue), 6 (purple), 10 (pink), and 12 (peach). (c) Overlap of the mode of binding of the best inhibitor (compound 6; shown in purple) with T4 (orange) at the T4 binding site of TTR. The interactions between TTR and the inhibitors in each of the minimum energy structure were evaluated using Clus-Pro online software^{46,47}. The side chains of protein residues that interact with a given ligand are shown. Residues involved in interactions within ≦4.5 A have only been shown.

Binding of BPEs to TTR are dominated by hydrophobic and electrostatic interactions with residues 15, 17, 108, 110, 117, 119, and 121. In spite of their common structural features, considerable differences were observed in the mode of binding to the T4 binding site. While the halogen binding pockets in TTR provide primarily a hydrophobic surface, conformational changes of its side chains facilitate additional hydrogen bonding interactions. The ligand-induced conformational changes of TTR not only allow energetically favorable Interactions between ligand and the protein but also stabilize the nonamyloidogenic tetramer of TTR against pH-mediated dissociation by the formation of inter-subunit hydrogen bonds.

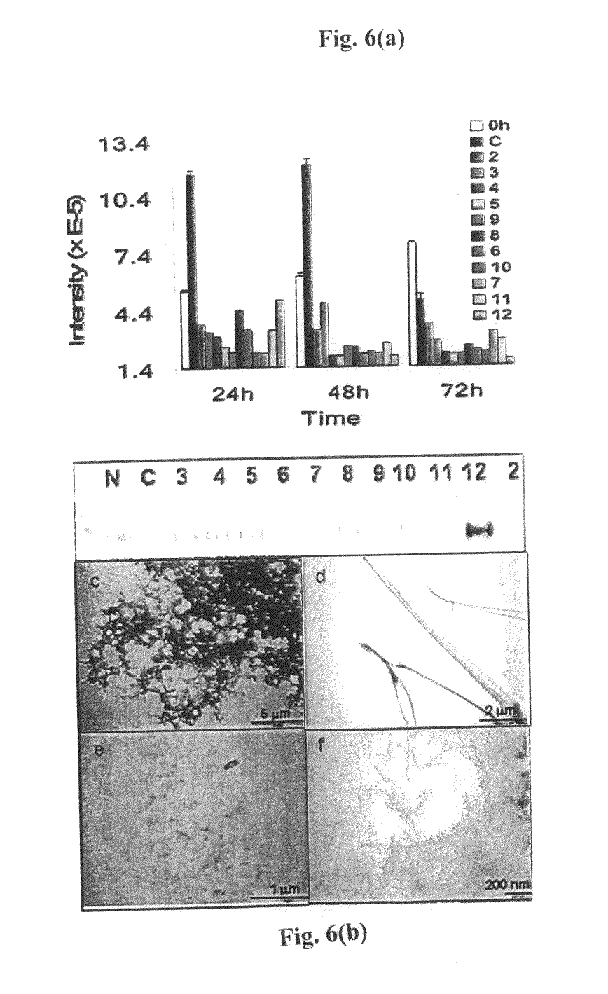

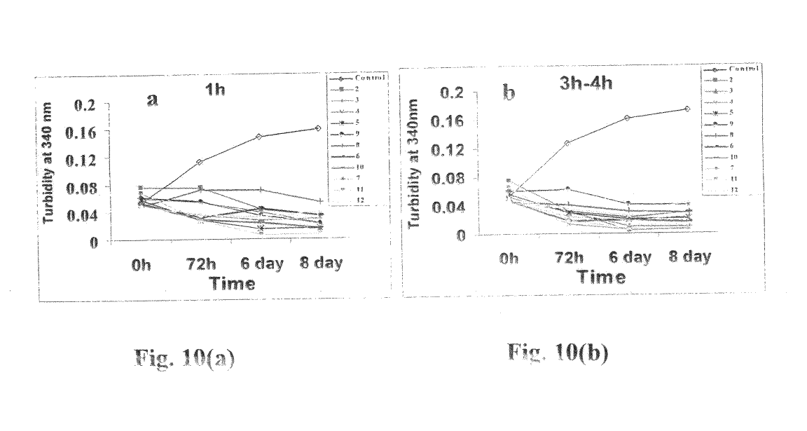

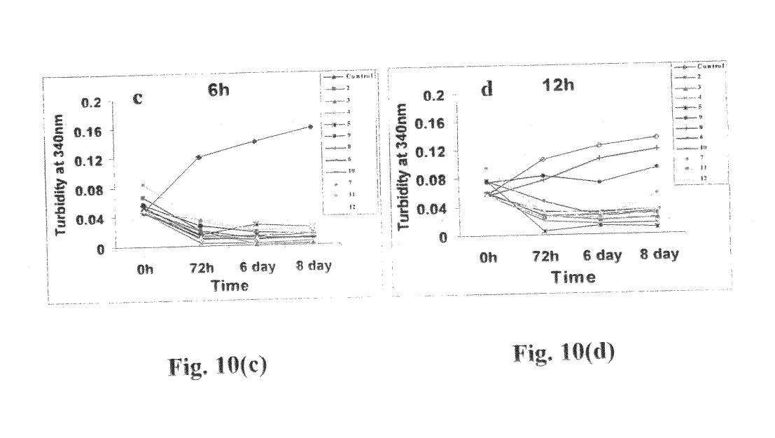

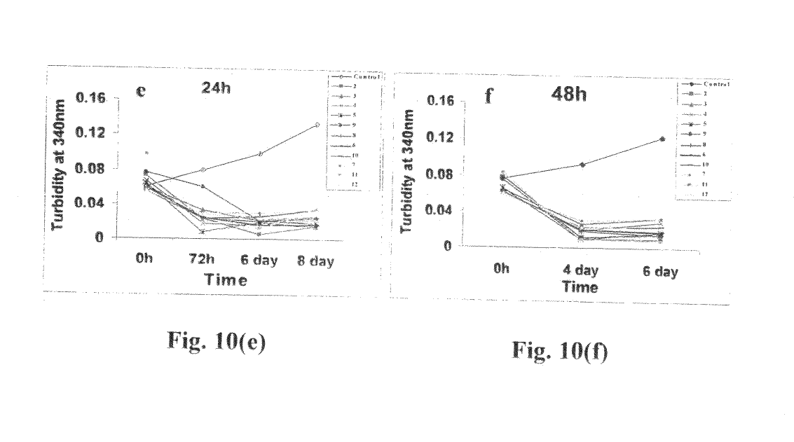

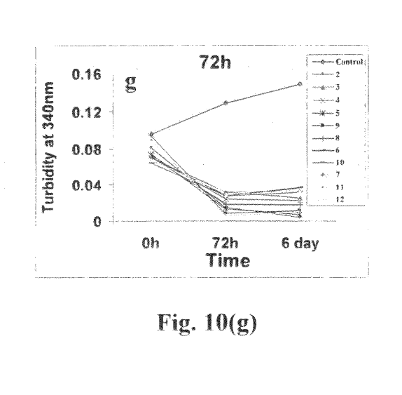

Fibril Disruption. The ability of these compounds to Inhibit TTR fibril elongation was studied. Their ability to disrupt preformed fibers was also examined. BPEs examined were not only able to inhibit the elongation of early fibrils but also exhibited disruption of the mature fibrils in a dose-dependent manner. Moreover, these compounds also disrupted the formation of various intermediates formed during TTR fibrilization. While single doses of inhibitors were adequate to prevent fibril elongation, multiple doses were required for the disruption of the preformed fibers. Disruption started after the second dose. The change in turbidity with time in the presence and the absence of compounds 2-12 is shown in FIG. 10. FIG. 10 shows the inhibition of fiber elongation and disruption of preformed TTR fibers by compound 2-12. Different intermediates formed at 1 h, 3 h, 6 h, 12 h, 24 h, 48 h and 72 h during the TTR fibrilization under acidic condition was incubated with inhibitors 2-12 (14.4 mM and 7.2 mM subsequent two doses at 24 h interval) in PBS. Change in turbidity at 340 nm was monitored for 8 days. The disruption of fibrils formed at 24, 48, and 72 h, viz after three dosages of these compounds, was also confirmed by Th-T fluorescence assay (FIG. 6a). FIG. 6 (a) shows Th-T fluorescence of TTR fibers after disruption by compounds 2-12. The TTR fiber formed at 24, 48, and 72 h were incubated with three doses (7.2 μM each) of compounds 2-12 for 15 days. The disruptions of preformed TTR fiber by these compounds were monitored by Th-T fluorescence by incubating samples with 50 μM of Th-T for 15 min at 37° C. The samples were excited at 450 nm, and emissions were monitored at 480 nm. Results are mean of three different experiments executed induplicates.

While the fluorescence intensity of Th-T in control samples increased with time, no enhancement in intensity was observed in the samples incubated for 15 days with compounds 2-12. All these compounds disrupt preformed TTR fibers as well. Compounds 4, 5, 6, 9, 10, and 12 were found more effective as fibril disrupters under the conditions used. Native-PAGE analysis of supernatants of above samples after 1 month clearly shows presence of abundant soluble protein compared to the untreated control (FIG. 6b). FIG. 6(b) shows Native-PAGE analysis of samples of TTR fibers disruption after 1 month of incubation with compounds 2-12 at 37° C. TTR fibers incubated with compounds 2-12 for 15 days were centrifuged, and supernatant was loaded on gel to see the amount of soluble protein present. Further, the effect of the compounds 2-12 on the ultrastructural properties of the TTR fiber was examined by TEM. FIG. 6 (d) shows the presence of abundant fibers (8-20 nm wide and 200-600 μm long) and fibrillar aggregates as compared to the control samples (FIG. 6 (c)).

FIGS. 6 (c)-(t) are Transmission electron micrograph of control sample showing fibrillar aggregates (1:2 diluted, 4.2K) (c), control sample showing full length fibers (1:100 diluted, 8.2 K) (d), fibers incubated with compounds 2-12 for 2 days were clearly disrupted (16.5 K), (e), magnified view of disrupted fibers (87K) (t). Further, the effect of the compounds 2-12 on the ultrastructural properties of the TTR fiber was examined by TEM. FIG. 6d shows the presence of abundant fibers (8-20 nm wide and 200-600 μm long) and fibrillar aggregates as compared to the control samples (FIG. 6c). FIG. 6 (c) shows transmission electron micrograph of control sample showing fibrillar aggregates (1:2 diluted, 4.2 K).

The fibrils with width of 14.4-21.6 μM were disrupted into fragments of 2-5 nm width and 20-100 μm long after the second dose of compounds 2-12. FIGS. 6e and 6f shows a representative picture of the fibers disrupted by compounds 2-12 subsequent to their second and third doses, respectively.

Besides, inhibiting the fibril formation, these compounds are also able to prevent the elongation of small prefibrillar species (FIG. 6). Fibril formation is completed within 72-80 h in case of TTR, and compounds 2-12 disrupt the fibers within this time limit, signifying the presence of structural motifs for the binding of these compounds.

The results presented above establish that the biphenyl ether template provides the shape and size complementarity to the TTR binding pocket, and carbonyl/carboxylic and chloride groups at position R₃ and R₁, respectively, are necessary for potentiating the interactions. The high potency of some of these compounds compared to any of the inhibitors described so far may be due to their structural complementarity to the T4 binding region of TTR as well as the solubility and stability of the complexes.

Moreover, binding of compounds 2-12 has an overall stabilizing effect on the TTR quaternary structure that surpasses the ability of other inhibitors. These molecules are able to prevent cytotoxicity induced by TTR in a neuronal cell culture. Further, they inhibit the fibril elongation at any step of the process as well as disrupt the preformed fibrils. The present study also shows that compounds 11 and 12 could be promising new structural templates for the design of potent inhibitors as therapeutic agents against TTR amyloidosis.

Although not being bound to any theory, it appears that the compounds studied here act by raising the energy barrier for dissociation by stabilizing the tetrameric ground state of TTR. The kinetic stabilization of the TTR tetramer is the most feasible strategy, since the identity of the species of the TTR amyloidogenesis pathway that induces toxicity still remains unknown. Although binding of these compounds to any amyloidogenic TTR mutants has not been tested, there are reports showing little or no structural changes in tetrameric conformation and T4 binding site in most of the TTR mutants. Hence, it can be assumed that molecules, which bind to wild type TTR, may also bind to these mutants. Therefore, a therapeutic strategy based on the development and administration of small molecules inhibitors could be explored for the prophylaxis of asymptomatic gene carriers. TTR has been shown to play an important role in keeping Aβ in soluble form. (Lin Liu Murphy, R. M. Kinetics of Inhibition of β-Amyloid Aggregation by Transthyretin, Biochemistry 2006, 45, 15702-15709). Hence, the increased stabilization of TTR by these compounds might also prevent the progress ion of other neurodegenerative disorders.

The compositions of the invention may be administered therapeutically or prophylactically to treat diseases associated with amyloid β fibril formation, aggregation, or deposition.

The compounds may be coupled to a (blood-brain barrier) transport vector.

The therapeutic compound may be administered to a subject in an appropriate carrier, for example, liposomes, or a diluent. Pharmaceutically acceptable diluents include saline, and aqueous buffer solutions.

The therapeutic compound may also be administered parenterally, intraperitoneally, intravenously, intramuscularly, intraspinally, or intracerebrally. Dispersions can be prepared in glycerol, liquid polyethylene glycols, and mixtures thereof and in oils or other suitable media.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like.

The percentage of the therapeutic compound in the compositions and preparations may, of course, be varied. The amount of the therapeutic compound in such therapeutically useful compositions is such that a suitable dosage will be obtained.

It will be appreciated by those skilled in the art that the amount of a compound required for use in treatment, modulation or inhibitation will vary with the nature of the condition being treated and the age and the condition of the patient and will be ultimately at the discretion of the attendant physician or other health care provider.

In accordance with the present invention, a compound described herein, and pharmaceutically acceptable salts thereof, may be administered orally or through inhalation as a solid, or may be administered intramuscularly or intravenously as a solution, suspension or emulsion. Alternatively, the compounds or salts may also be administered by inhalation, intravenously or intramuscularly as a liposomal suspension.

Pharmaceutical formulations are also provided which are suitable for administration as an by inhalation, intranasally, or transmucosally.

Active compounds are administered at a therapeutically effective dosage sufficient to inhibit amyloid deposition in a subject. By “therapeutic” or “drug” is meant an agent having a beneficial ameliorative or prophylactic effect on a specific disease or condition in a living human or non-human animal.

Certain embodiments of the present compounds can contain a functional group, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed.

The following examples are intended to Illustrate but not to limit the present invention

### EXAMPLES

### Preparation of Inhibitors

The chemical library was purchased from Chemical Diversity Labs Inc., San Diego, Calif. Synthesis of substituted BPE has recently was reported. Chhibber, M., Kumar, G.; Parasuraman, P.; Ramya, T. N., Surolia, N.; Surolia, A. Novel diphenyl ethers: design, docking studies, synthesis and inhibition of enoyl ACP reductase of Plasmodium falciparum and Escherichia coli Bioorg. Med. Chem. 2006, 14 (23), 8086-8098. Triclosan was obtained from Kumar Chemicals, Bangalore, India. The schemes used for the synthesis of the BPEs are shown above. Information. Stock solutions (10 mM) of the compounds used were prepared in DMSO. All compounds diluted from the stock solution were soluble at 50-100 μm concentrations in aqueous buffer used for assessing their inhibitory potencies in assays to monitor TTR amyloidosis.

Synthesis of Compounds

General Melting points were determined with Buchi apparatus and are uncorrected. Microanalyses were performed on an automated C, H, N analyzer. Mass analysis was done using Electrospray mass spectrometer; Gas chromatography-coupled mass spectrometer, and high-resolution mass spectrometer. 1 Hand 13C NMR spectral analysis were performed on 300, 400, and 75, 100 MHz spectrometer, respectively, with tetramethylsilane as the internal standard (8 ppm). The following abbreviations were used to explain the multiplicities: s, singlet, d, doublet, t, triplet; dd, double doublet; m, multiplet, br, broad. Solvents and reagents were purified according to standard laboratory technique.

All compounds, except 18, 6, 20, were prepared by the reported procedure and the analytical data has been compiled in Table-A.

4-(2′,4′-Dinitrophenoxy)-3-methoxybenzyl chloride (18): To an ice cooled solution of 19 (200 mg, 0.62 mmol) and pyridine (0.1 ml, 1.56 mmol) in diethyl ether (20 ml) was added drop wise PC13 (0.03 ml, 0.31 mmol) and the reaction mixture stirred overnight. On completion of reaction (TLC) it was quenched with water and extracted with diethyl ether (15 ml×3), washed with water (10 ml×2), brine (10 ml) and dried over Na2S0a. The evaporation of the organic solvent gave crude product which was purified using Si02 column chromatography and solvent (toluene) to afford pure 18 (150 mg) in 71% yield.

3-Methoxy-4-phenoxybenzyl chloride (6): Following the procedure detailed for 18 above compound 6 was isolated as oily liquid 78% yield.

4-(2′,4′-Dinitrophenoxy)-3-methoxybenzyl acetate (20): To an ice cooled solution of 19 (320 mg, 1.0 mmol) and pyridine (0.15 ml, 2.0 mmol) in dichloromethane (20 ml) was added acetic anhydride (0.14 ml, 1.5 mmol) and the reaction mixture stirred overnight.

On completion of reaction (TLC) it was quenched with water and extracted with dichloromethane (15, ml×3), washed with water (10 ml×2) and dried over Na2SO4. The evaporation of the organic solvent gave crude product which was purified using SiO2 column chromatography and solvent (toluene) to afford pure 20 (342 mg) in 95% yield.

Expression and Purification of WT-TTR. TTR cloned in pMMHa vector was a kind gift of Dr. P. Raghu from National Institute of Nutrition, Hyderabad, India. Protein expression and purification was performed as reported earlier. Lashuel, H. A.; Wurth, C., Woo, L., Kelly, J. W. The most pathogenic transthyreti1 variant, L55P, forms amyloid fibrils under acidic conditions and protofilaments under physiological conditions. Biochemistry 1999, 38 (41), 13560-135 revised 73.

Protein concentrations were measured by absorbane at 280 nm Protein purity was assessed by SDS-PAGE, and its mass was confirmed by electrospray ionization mass spectrometry (ESI-MS).

### Example 1

Stagnant Acid-Mediated TTR Aggregation Assay. The efficacy of compounds 1-12 was determined by stagnant acid-mediated turbidity assay at 340 nm using Tecan GENios microplate reader.

For stagnant aggregation assays a series of eppendorf tubes containing 7.2 μM tetramer (0.4 mg/ml) of TTR in 5 mM sodium phosphate, 100 mM KCl, 1 mM EDTA, pH 7 were incubated with inhibitor 7.2 (1:1) and 21.6 μM (1:3) (DMSO 1%) in 0.5 ml at 25° C. After 1 h, the samples were diluted with 0.5 ml of 200 mM sodium acetate buffer containing 100 mM KCl and 1 mM EDTA. Samples after mild vortexing were incubated at 37° C. for the desired amount of time without stirring to evaluate the efficacy of the inhibitors. The extent of aggregation was probed by turbidity measurements at 340 nm using Tecan GENios microplate reader. Single time point samples in eppendorfs (72 h) were vortexed for 5 sec immediately before the measurement to quantify fibril formation. The extent of TTR fibril formation in the absence of inhibitor was defined to be 100%. Inhibitors were tested in the absence of TTR to evaluate their intrinsic absorbance and confirm that the inhibitor was soluble over the course of the assay (i.e. does not contribute to the turbidity, none of the biphenyl ethers show 00>0.05 at 340 nM at the maximum concentration, used except thyroxine). In another assay inhibitors were buffered in 0.1 M sodium acetate buffer containing 100 mM KCl and 1 mM EDTA pH 4.4 and TTR was added directly to this solution and incubated at 37° C. for 72 h to assess; the affinity of these inhibitors for TTR under fibrillation condition. All experiments were done in triplicate. The stoichiometry of protein inhibitor binding was determined by HPLC as described by Green et al for IC₅₀ value 7.2 μM of TTR incubated with 1-50 μM of inhibitors and assayed as above. The IC₅₀ was determined by plotting mean of % inhibition vs log [1] and fitted to sigmoid equation:

y=a/l+e^{-(x-x-o/b)}

The solution of TTR tetramer (7.2 μM; 0.4 mg/mL) in 5 mM sodium phosphate, 100 mM KCl, 1 mM EDTA, pH 7, were incubated with inhibitor at 7.2 and 21.6 μM, viz, TTR tetramer to inhibitor ratio of 1:1 and 1:3, respectively, in 1% DMSO in a total volume of 0.5 ml. The experiments were conducted at 25° C. After 1 h, the samples were diluted with 0.5 mL of 200 mM sodium acetate buffer pH 4.4 containing 100 mM KCl and 1 mM EDTA, vortexed, and incubated at 37° C. for 72 h. In another assay, inhibitors were buffered in the same pH 4.4 buffer, and TTR was added directly to this solution and incubated at 37° C. for 72 h to assess the affinity of these inhibitors for TTR under fibrillation condition. The IC₅₀ of inhibition of these inhibitors were studied by incubating the TTR (7.2 μM) with 0.5-50 μM of inhibitors, and the IC₅₀ values were calculated graphically. The stoichiometry of the binding of the inhibitors was determined by HPLC as described by Green et al., N. S. Palaninathan, S, L, Sacchettini, J. e. Kelly, J W. Synthesis and characterization of potent bivalent amyloidoisis inhibitors that bind prior to transthyretin tetramerization, J. An Chem. Soc. 2003, 125, 13404-13414. Dissociation constant of binding of these compounds to TTR was determined by fluorescence titration, and data were fitted into the Adair equation for two bind mg.

Determination of binding constant: The dissociation constant of compound with TTR was measured by fluorescence titration on Jobin Yvon Fluoromax spectrofluorometer using an excitation slit width of 2 nm and emission of 5 nm. Small aliquots of 1 mM ligands solutions were successively added to the 2 mM TTR in 100 mM sodium acetate buffer pH 4.4 at 200 C under constant stirring. After each addition sample was left for 2 min. Samples were exited at 295 nm and emission was recorded at 342 nm. Average of 10 measurements was taken. Readings were corrected for buffer blank, dilution and inner filter effect. The inner filter effect was corrected by using the following equation,

Fc=F antilog [(Aex+Aem)/2]

Where Fc is the corrected fluorescence and F is the measured one, Aex and Aem are the absorbance of the reaction solution at the excitation and emission wavelength, respectively.

From the titration data the dissociation constant was determined by fitting data in following Adair equation for two binding sites-

Y=(2*KI*x+KI*K2*(x2))/(1+KI*x+KI*K2*(x2))

Data for compound 9 and 12 fitted well to one site of Adair equation.

All experiments were done In triplicate.

### Example 2

Time Course of Fibril Formation. The kinetics of inhibition was studied by incubating 7.2 pM TTR with 21.6 pM of different inhibitors in 100 pl of 5 mM phosphate buffer pH 7.2 for 30 min in a 96 microwell plate. After 30 mM 100 wl of 0.2 M sodium acetate buffer pH 4.4 containing 1 mM EDTA and 100 mM KCl was added. The difference in turbidity was assessed by measuring the change in absorbance at 340 nm on Tecan GEnios microplate reader using Magellan software. For kinetics, parameters were set for 30 cycles at 37° C. at the interval of 5 min each and 2 min orbital shaking (low speed) between cycles and 10 sec shaking (normal speed) before measurement. All experiments were performed in triplicates. Change in absorbance at 340 nm was plotted against time in the presence and absence of inhibitors initial rate of fibril formation was calculated from the slope by linear fitting of the data.

All experiments were performed three times in triplicates. Change in absorbance (mean of three different experiments) at 340 nm was plotted against time in the presence and absence of inhibitors. Initial rate of fibril form action was calculated from the value of the slope by linear fitting of the change in absorbance at 340 nm as a function of time.

### Example 3

Congo Red Binding. The amount of bound Congo red was estimated as reported earlier Lashuel, H. A., Wurth C. Woo, L., Kelly, J. W. The most pathogenic transthyretin variant, LS5P, forms amyloid fibrils under acidic conditions and protofilaments under physiological conditions. Biochemistry 1999, 38 (41), 13560-135 revised 73 using the equation, moles of Congo red bound/L of amyloid suspension=A₅₄₀(nm)/25295−A₄₇₇(nm)/46306.

### Example 4

Thioflavin-T Fluorescence. The thioflavin-T (ThT) binding assays were performed in a Jobin Yvon Fluoromax spectrofluorimeter using an excitation slit width of 2 nm and emission slit width of 5 nm. Le Vine, H. Quantification of β-Sheet Amyloid Fibril Structures with Thioflavin T Methods Enzymol. 1999, 309, 274-284. In brief, samples were incubated with 50 μM of Th-T for 15 min at 37° C. in the dark. The samples were excited at 450 nm, and emissions were monitored at 480 nm. The inner filter effect was corrected by using the following equation,

Fc=Fx antilog [(Aₑₓ+A_{cm})/2]

where Fc is the corrected fluorescence and F is the measured one, Aₑₓ and A_{cm} are the absorbances of the reaction solution at the excitation and emission wavelengths, respectively. All experiments were done in triplicate.

### Example 5

Intrinsic Fluorescence of TTR. For monitoring the intrinsic fluorescence of TTR, 0.2 mL of 3.6 μM TTR solutions was incubated with 10.8 μM concentration of a given inhibitor at pH 7.2 and 4.4. Samples were excited at 290 nm, and emission was recorded between 310 and 370 nm.

Tryptophan fluorescence was recorded on Jobin Yvon Horiba fluorimeter attached to a computer with excitation and emission monochromator slit widths of 2 nm and 3 nm, respectively. Quartz cuvette (0.2 ml) was used for the study.

All the experiments were performed at least three times and typically in duplicates.

### Example 6

Urea Denaturation. TTR (1.8 was incubated with 5.4 μM inhibitors at 37° C. for 30 min in 400 μL of 50 mM phosphate buffer containing 1 mM EDTA, 1 mM DTT and 100 mM KCf. After 30 min, 600 μL of 10 M urea stock in the same buffer was added to bring the final concentration of urea to 6 M. Tryptophan fluorescence was measured in the range of 310-370 nm up to 144 h as described above and corrected for inner filter effect. The results are the mean of three different experiments done in duplicates.

### Example 7

Glutaraldehyde Cross-Linking. The quaternary structural changes of TTR at pH 4.4 in the presence and absence of all these inhibitors was monitored by glutaraldehyde cross-linking and SDS-PAGE. Colon, W.; Kelly, J W. Partial denaturation of trans thy ret in is sufficient for amyloid fibril formation in vitro. Biochemistry 1992, 31,8654-8660.

A 200 PI solution of TTR (0.4 mg/ml) was incubated at pH 4.4 in the presence and absence of 10.8 PM inhibitors at 37° C. for 72 h to 15 days. For gluteraldehyde crosslinking 200 [1 solution was taken after Mays. Following incubation, 70˜tl of 0.5 M phosphate buffer, pH 7.5, as added to each sample for neutralization. Immediately, 200 gl aliquots were taken into fresh vials containing 8 [1 of 25% glutaraldehyde. The cross-linking was carried out for exactly 6 min and further reaction was stopped by the addition of 10 pl of NaBH4 (7 g/100 ml 0.1 M NaOH). To the reaction mixtures (5081) 50˜1 of 5% SDS sample buffer was added, and the mixture boiled samples were then loaded onto 12.5% SDS-PAGE. TTR incubated at pH 7.5 was used as a control and processed as described above except for the neutralization step. The gels were developed with Coomassie brilliant blue stain and analyzed by using BioRad GS-71 0 imaging densitometer.

### Example 8

Mass Spectrometry. Inhibition of fibril formation by these compounds was further confirmed by mass spectrometry. Reaction mixture from aggregation assay was taken at 0.72 hand 7-15 days centrifuged at 15,000 rpm for 30 min to remove all aggregates and then filtered through 80 kDa cutoff membrane. 1 pL of the supernatant solution was mixed with 1 pL of saturated solution of sinapinic acid (3-(4-hydroxy-3,5-dimethoxy-phenyl)prop-2-enoic acid) in ethanol containing 0.5% trifluoroacetic acid. This mixture was loaded on to the target plate. Mass was recorded using Bucker MALDI-TOF instrument. TTR frozen at −20° C., pH 7.4 was used as positive control.

Aliquots of the samples of TTR kept for assaying fibril formation in the absence (0 h sample) and presence of compounds 1-12 at 3-15 days were centrifuged at 15 000 rpm for 30 min and then filtered through 80 kDa cuff-off membranes. Subsequently, 1 μL of the filtrate was mixed with 1 μL of matrix, 3-(4-hydroxy-3,5-dimethoxy-phenyl)prop-2-enoic acid in ethanol containing 0.5% trifluoroacetic acid and loaded on to the target plate. Mass spectra were recorded using Brucker MALDI-TOF instrument. TTR frozen at −20° C., pH 7.4, was used as the positive control.

### Example 9

Cell Culture and Cytotoxicity Inhibition Assay. The effect of TTR and compounds 2-12 on adherent human neuro2a cell line was monitored as described earlier. Surolia, I., Reddy, G B., Sinha, S. Hierarchy and the mechanism of fibril formation in ADan peptides. J. Neurochem. 2006, 99, 537-548.

The adherent human neuro2a cell line, was grown in 75 cm2 flasks in RPMI-1640 (Sigma, USA), supplemented with 10°/a FBS, 1 mM Hepes buffer/2 mM L-glutamine/100 units/ml penicillin/100 gg/ml streptomycin (complete cell media), and incubated at 37° C. in a 5% CO2 atmosphere. For cell toxicity assay the 25 μM of filter sterilized WTTR was incubated with 50 pM of compounds 2-12 in PBS for 1 h at 30° C. to facilitate the interaction of the compounds with TTR. After 1 h samples were lyophilized. These samples were reconstituted in complete cell culture medium just before the experiment. The TTR and compounds were also diluted with equal volumes of cell assay media to serve as controls for intrinsic TTR and drug cytotoxicity, respectively. Neuro2a cells (80-90% confluent) were plated into 96-well plates in complete cell media at a density of 100,000 cells per well and were incubated overnight at 370° C. in a 5% CO2 atmosphere. The media from the cells was removed and 100 μl of the TTR samples (TTR, TTR with compounds, compounds alone, or cell media alone) were immediately added to each well. The wells containing cells and wells without cells received 100 μl of media without TTR to serve as controls and blanks, respectively. After addition of TTR samples (or media), the plates were incubated at 37° C. in a 5% CO2 atmosphere for 48 h. To assay the viability ten microliters per well of 3-[4,5dimethylthiazol-2-yl]-2,5diphenyl tetrazolium bromide (MIT) (5 mg/ml in complete medium) were added to the wells (samples, blanks, and controls), and the plates were incubated for 3-4 hat 370° C. Then, 125 μl per well of lysis solution (5.0% HCl in isopropanol) was added, and the plates were incubated overnight at 37° C. to solubilize the formazan produced, which was quantified by measuring OD at 570 nm in a 96-well plate reader (Tecan GENios). All of the assays were carried out twice in triplicate. The results were calculated as % viability/% cytotoxicity.

All the assays were carried out twice in triplicates. The results were calculated as (% viability)/(% cytotoxicity).

### Example 10

Molecular Docking. The MOE-200S (Molecular Operating Environment) software was used to perform docking. The rebuilt algorithms were followed. Before docking, the coordinates of the biphenyl ethers were energy minimized using the force field MMFF94. Prior to docking studies all the water molecules have been removed from TTR (POB code le4 h) structure. Molecular docking with the TTR tetramer was done with each of the mentioned compounds. From a cluster of 100 docked structures, the one with the minimum energy was considered for further studies. Further, we evaluated the Interactions between TTR and the inhibitors in each of the minimum energy structures using LPC-CSU online software. Sorokine, A.; Prilusky, J.; Abola, E. E.; Edelman, M. Automated analysis of interatomic contacts in proteins. Bioinformatics 1999, 15, 327-332. We overlapped these docked structures of compounds 1-12 with the crystal structure of T4 bound to human TTR (POB code 2ROX) to validate the accuracy of docking experiments.

### Example 11

Fibril Disruption Assay. A 14.4 mM concentration of TTR was incubated with 100 mM sodium acetate pH 4.4 containing 1 mM EOTA, 0.1 M KCl for 0, 13, 6, 12, 24, 48, and 72 h at 37° C. After indicated time points, the fibers (7.2 μM) were incubated with the compounds 2-12 (14.4 μM) in PBS at 37° C. The disruption was followed for 7 days by turbidity measurements at 340 nm and Th-T fluorescence. A second dose of the inhibitor (7.2 μM) was added after 24 h of incubation, and samples were incubated and monitored further for 5-6 days. After 15-20 days of incubation, samples were examined by transmission electron microscopy (TEM). All experiments were done thrice in triplicates each time.

### Example 12

Transmission Electron Microscopy. The samples were vortexed and immediately absorbed to glow discharged carbon-coated 200 mesh copper grids as such or diluted to 1:2-1:100 fold with 0.15M NaCl in case of control and washed with deionized water. In case of samples with inhibitors were centrifuged at 10000 rpm and the resulting pellets was resupended in to 10 ml of 0.15M NaCl and loaded on grid. Negative staining was done by incubating grids in 3% uranyl acetate for 45 seconds and dried under infrared light. The grids were visualized with a FEI TECNAI G2 at 120 kV and exhaustively examined. The picture was captured using Mega View III camera and analyzed using AnalySIS Software from Imaging System GmbH.

1. A method of treating, modulating or preventing an amyloid related disease comprising administering an effective amount of a derivative of biphenylether, a compound included in Table 2 or a pharmaceutically acceptable salt thereof to a subject in need thereof.

2. A method of treating, modulating or preventing an amyloid related disease comprising administering a composition comprising an effective amount of a derivative of a biphenylether, a compound included in Table 2 or a pharmaceutically acceptable salt thereof to a subject in need thereof.

3. The method of claim 1, wherein the derivative of biphenylether, compound included in Table 2 or a pharmaceutically acceptable salt thereof slows the rate of amyloid β fibril formation or deposition; lessens the degree of amyloid β deposition; inhibits, reduces, or prevents amyloid β fibril formation; inhibits neurodegeneration or cellular toxicity induced by amyloid β; inhibits amyloid β induced inflammation; or enhances the clearance of amyloid β from the brain.

4. The method of claim 1, wherein the derivative of biphenylether, compound included in Table 2 or a pharmaceutically acceptable salt thereof stabilizes or slows cognitive function in a patient with brain amyloidosis.

5. The method according to claim 4, wherein the brain amyloidosis is Alzheimer's disease or cerebral amyloid angiopathy.
